SEQUENCE LISTING (62 sequences)

SEQ ID NO: 1 - protein, 295 aa, Sus scrofa
TYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLSLSRVPEIEDMEISLPNIHYFNIDMSKMGLINKEEVLLPLDNPYGKITGTVKRKLS

SEQ ID NO: 2 - protein, 298 aa, Sus scrofa
TYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 3 - protein, 301 aa, Papio anubis
MADYHNNYKKNDELEFVRTGYGKDMVKVLHIQRDGKYHSIKEVATSVQLTLSSKKDYLHGDNSDIIPTDTIKNTVHVLAKFKGIKSIEAFGVNICEYFLSSFNHVIRAQVYVEEIPWKRLEKNGVKHVHAFIHTPTGTHFCEVEQLRSGPPVIHSGIKDLKVLKTTQSGFEGFIKDQFTTLPEVKDRCFATQVYCKWRYHQCRDVDFEATWGTIRDLVLEKFAGPYDKGEYSPSVQKTLYDIQVLSLSRVPEIEDMEISLPNIHYFNIDMSKMGLINKEEVLLPLDNPYGKITGTVKRKLS

SEQ ID NO: 4 - protein, 304 aa, Sus scrofa
MAHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGRITGTVKRKLTSRL

SEQ ID NO: 5 - protein, 304 aa, Canis lupus
MAHYHNDYKKNDEVEFVRTGYGKDMVKVLHIQRDGKYHSIKEVATSVQLTLSSKKDYVYGDNSDIIPTDTIKNTVHVLAKFKGIKSIETFAMNICEHFLSSFNHVIRAQVYVEEVPWKRFEKNGVKHVHAFIHNPTGTHFCEVEQMRSGPPVIHSGIKDLKVLKTTQSGFEGFIKDQFTTLPEVKDRCFATKVYCKWRYHQGRDVDFEATWDTVRDIVLEKFAGPYDKGEYSPSVQKTLYDIQVHSLSRVPEMEDMEISLPNIHYFNIDMSKMGLINKEEVLLPLDNPYGRITGTAKRKLASKL

SEQ ID NO: 6 - protein, 304 aa, Bos taurus
MAHYHNDYQKNDEVEFVRTGYGKDMVKVLHIQRDGKYHSIKEVATSVQLTLNSRREYLHGDNSDIIPTDTIKNTVQVLAKFKGIKSIETFAMNICEHFLSSFNHVIRVQVYVEEVPWKRFEKNGVKHVHAFIHTPTGTHFCEVEQLRSGPPVIHSGIKDLKVLKTTQSGFEGFLKDQFTTLPEVKDRCFATQVYCKWRYHQGRDVDFEATWEAVRGIVLKKFAGPYDKGEYSPSVQKTLYDIQVLSLSQLPEIEDMEISLPNIHYFNIDMSKMGLINKEEVLLPLDNPYGRITGTVKRKLTSRL

SEQ ID NO: 7 - protein, 300 aa, synthetic construct
AHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLSLSRVPEIEDMEISLPNIHYFNIDMSKMGLINKEEVLLPLDNPYGKITGTVKRKLS

SEQ ID NO: 8 - protein, 301 aa, synthetic construct
MAHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLSLSRVPEIEDMEISLPNIHYFNIDMSKMGLINKEEVLLPLDNPYGKITGTVKRKLS

SEQ ID NO: 9 - DNA, 903 bp, synthetic construct
  source 1..903: mol_type = other DNA
gcccactacaggaatgattacaagaagaacgatgaggtggagttcgtgagaaccggctacggcaaggacatgatcaaggtgctgcacatccagagagacggcaagtaccacagcatcaaggaggtggccacctccgtgcagctgacactgagctccaagaaggattacctgcacggcgataatagcgatgtgatccccacagacaccatcaagaacacagtgaacgtgctggccaagtttaagggcatcaagtccatcgagacatttgccgtgaccatctgtgagcacttcctgtccagctttaagcacgtgatcagagcccaggtgtacgtggaggaggtgccttggaagaggttcgagaagaatggcgtgaagcacgtgcacgcctttatctacacacccaccggcacccacttctgcgaggtggagcagatcagaaatggccctcccgtgatccactccggcatcaaggacctgaaggtgctgaagaccacccagagcggcttcgagggcttcatcaaggaccagttcaccaccctgcccgaggtgaaggatagatgcttcgccacacaggtgtactgtaagtggagataccaccagggcagagacgtggacttcgaggccacatgggacacagtgagaagcatcgtgctgcagaagtttgccggcccttacgacaagggcgagtacagcccctctgtgcagaagaccctctatgatatccaggtgctctccctgagccgagttcctgagatagaagatatggaaatcagcctgccaaacattcactacttcaatatagacatgtccaaaatgggtctgatcaacaaggaagaggtcttgctgccattagacaatccatatggaaaaattactggtacagtcaagaggaagttgtcttga

SEQ ID NO: 10 - DNA, 903 bp, synthetic construct
  source 1..903: mol_type = other DNA
atggcccactacaggaatgattacaagaagaacgatgaggtggagttcgtgagaaccggctacggcaaggacatgatcaaggtgctgcacatccagagagacggcaagtaccacagcatcaaggaggtggccacctccgtgcagctgacactgagctccaagaaggattacctgcacggcgataatagcgatgtgatccccacagacaccatcaagaacacagtgaacgtgctggccaagtttaagggcatcaagtccatcgagacatttgccgtgaccatctgtgagcacttcctgtccagctttaagcacgtgatcagagcccaggtgtacgtggaggaggtgccttggaagaggttcgagaagaatggcgtgaagcacgtgcacgcctttatctacacacccaccggcacccacttctgcgaggtggagcagatcagaaatggccctcccgtgatccactccggcatcaaggacctgaaggtgctgaagaccacccagagcggcttcgagggcttcatcaaggaccagttcaccaccctgcccgaggtgaaggatagatgcttcgccacacaggtgtactgtaagtggagataccaccagggcagagacgtggacttcgaggccacatgggacacagtgagaagcatcgtgctgcagaagtttgccggcccttacgacaagggcgagtacagcccctctgtgcagaagaccctctatgatatccaggtgctctccctgagccgagttcctgagatagaagatatggaaatcagcctgccaaacattcactacttcaatatagacatgtccaaaatgggtctgatcaacaaggaagaggtcttgctgccattagacaatccatatggaaaaattactggtacagtcaagaggaagttgtct

SEQ ID NO: 11 - protein, 20 aa, synthetic construct
MYRMQLLSCIALSLALVTNS

SEQ ID NO: 12 - protein, 24 aa, synthetic construct
MAVMAPRTLVLLLSGALALTQTWA

SEQ ID NO: 13 - protein, 35 aa, synthetic construct
MSSWSRQRPKSPGGIQPHVSRTLFLLLLLAASAWG

SEQ ID NO: 14 - protein, 20 aa, synthetic construct
MEPWPLLLLFSLCSAGLVLG

SEQ ID NO: 15 - protein, 27 aa, synthetic construct
MDPPRPALLALLALPALLLLLLAGARA

SEQ ID NO: 16 - protein, 20 aa, synthetic construct
MMWPMHTPLLLLTALMVAVA

SEQ ID NO: 17 - protein, 26 aa, synthetic construct
MTAAAGSAGRAAVPLLLCALLAPGGA

SEQ ID NO: 18 - protein, 27 aa, synthetic construct
MAPHRPAPALLCALSLALCALSLPVRA

SEQ ID NO: 19 - protein, 16 aa, synthetic construct
MPLLLLLLLLPSPLHP

SEQ ID NO: 20 - protein, 18 aa, synthetic construct
MQPILLLLAFLLLPRADA

SEQ ID NO: 21 - protein, 22 aa, synthetic construct
MASRLTLLTLLLLLLAGDRASS

SEQ ID NO: 22 - protein, 22 aa, synthetic construct
MCPARSLLLVATLVLLDHLSLA

SEQ ID NO: 23 - protein, 18 aa, synthetic construct
MHSSALLCCLVLLTGVRA

SEQ ID NO: 24 - protein, 18 aa, synthetic construct
MGFWILAILTILMYSTAA

SEQ ID NO: 25 - protein, 27 aa, synthetic construct
MGTGGRRGAAAAPLLVAVAALLLGAAG

SEQ ID NO: 26 - protein, 21 aa, synthetic construct
MSLLVVSMACVGFFLLQGAWP

SEQ ID NO: 27 - protein, 18 aa, synthetic construct
MFLLLTALQVLAIAMTQS

SEQ ID NO: 28 - protein, 19 aa, synthetic construct
MKFTTLLFLAAVAGALVYA

SEQ ID NO: 29 - protein, 28 aa, synthetic construct
MWEAQFLGLLFLQPLWVAPVKPLQPGAE

SEQ ID NO: 30 - DNA, 15 bp, Homo sapiens
  source 1..15: mol_type = other DNA
cagcacgagctgctc

SEQ ID NO: 31 - DNA, 15 bp, Homo sapiens
  source 1..15: mol_type = other DNA
cttcttcttcctctt

SEQ ID NO: 32 - DNA, 15 bp, Homo sapiens
  source 1..15: mol_type = other DNA
tcatgccggtgctgg

SEQ ID NO: 33 - DNA, 15 bp, Homo sapiens
  source 1..15: mol_type = other DNA
accaaatggagaaca

SEQ ID NO: 34 - DNA, 15 bp, Homo sapiens
  source 1..15: mol_type = other DNA
tcagatgtgcagaca

SEQ ID NO: 35 - DNA, 11 bp, Homo sapiens
  source 1..11: mol_type = other DNA
ccagagcctgg

SEQ ID NO: 36 - DNA, 20 bp, Homo sapiens
  source 1..20: mol_type = other DNA
cattgctaacaatgtccact

SEQ ID NO: 37 - DNA, 10 bp, Homo sapiens
  source 1..10: mol_type = other DNA
gctgaagctg

SEQ ID NO: 38 - DNA, 20 bp, Homo sapiens
  source 1..20: mol_type = genomic DNA
actggagttctgtgacagga

SEQ ID NO: 39 - DNA, 10 bp, Homo sapiens
  source 1..10: mol_type = genomic DNA
tcttcctctt

SEQ ID NO: 40 - DNA, 741 bp, Coxsackie virus B3
  source 1..741: mol_type = genomic DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 41 - DNA, 249 bp, Anabaena sp.
  source 1..249: mol_type = genomic DNA
aaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 42 - DNA, 51 bp, Anabaena sp.
  source 1..51: mol_type = genomic DNA
aaaatccgttgaccttaaacggtcgtgtgggttcaagtccctccaccccca

SEQ ID NO: 43 - DNA, 40 bp, Anabaena sp.
  source 1..40: mol_type = genomic DNA
aaaatccgttgaccttaaacggtcgtgtgggttcaagtcc

SEQ ID NO: 44 - DNA, 30 bp, Anabaena sp.
  source 1..30: mol_type = genomic DNA
aaaatccgttgaccttaaacggtcgtgtgg

SEQ ID NO: 45 - DNA, 20 bp, Anabaena sp.
  source 1..20: mol_type = genomic DNA
aaaatccgttgaccttaaac

SEQ ID NO: 46 - DNA, 15 bp, Anabaena sp.
  source 1..15: mol_type = genomic DNA
agacgctacggactt

SEQ ID NO: 47 - DNA, 10 bp, Anabaena sp.
  source 1..10: mol_type = genomic DNA
ctacggactt

SEQ ID NO: 48 - DNA, 15 bp, synthetic construct
  source 1..15: mol_type = other DNA
agacgctacggagtt

SEQ ID NO: 49 - DNA, 15 bp, synthetic construct
  source 1..15: mol_type = other DNA
agacgctacggattt

SEQ ID NO: 50 - DNA, 1827 bp, synthetic construct
  source 1..1827: mol_type = other DNA
aaaatccgttgaccttaaacggtcgtgtgggttcaagtccctccacccccacgccggaaacgcaatagccgttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaagccaccatgtacaggatgcaactcctgtcttgcattgcactaagtcttgcacttgtcacgaattcggcccactacaggaatgattacaagaagaacgatgaggtggagttcgtgagaaccggctacggcaaggacatgatcaaggtgctgcacatccagagagacggcaagtaccacagcatcaaggaggtggccacctccgtgcagctgacactgagctccaagaaggattacctgcacggcgataatagcgatgtgatccccacagacaccatcaagaacacagtgaacgtgctggccaagtttaacggcatcaagtccatcgagacatttgccgtgaccatctgtgagcacttcctgtccagctttaagcacgtgatcagagcccaggtgtacgtggaggaggtgccttggaagaggttcgagaagaatggcgtgaagcacgtgcacgcctttatctacacacccaccggcacccacttctgcgaggtggagcagatcagaaatggccctcccgtgatccactccggcatcaaggacctgaaggtgctgaagaccacccagagcggcttcgagggcttcatcaaggaccagttcaccaccctgcccgaggtgaaggatagatgcttcgccacacaggtgtactgtaagtggagataccaccagggcagagacgtggacttcgaggccacatgggacacagtgagaagcatcgtgctgcagaagtttgccggcccttacgacaagggcgagtacagcccctctgtgcagaagaccctctatgatatccaggtgctctccctgagccgagttcctgagatagaagatatggaaatcagcctgccaaacattcactacttcaatatagacatgtccaaaatgggtctgatcaacaaggaagaggtcttgctgccattagacaatccatatggaaaaattactggtacagtcaagaggaagttgtcttcaagactgtgacggctattatgcgttaccggcgagacgctacggactt

SEQ ID NO: 51 - DNA, 1106 bp, synthetic construct
  source 1..1106: mol_type = other DNA
aaaatccgttgaccttaaacggtcgtgtgggttcaagtccctccacccccacgccggaaacgcaatagccgactggagttctgtgacaggagccaccatgtacaggatgcaactcctgtcttgcattgcactaagtcttgcacttgtcacgaattcggcccactacaggaatgattacaagaagaacgatgaggtggagttcgtgagaaccggctacggcaaggacatgatcaaggtgctgcacatccagagagacggcaagtaccacagcatcaaggaggtggccacctccgtgcagctgacactgagctccaagaaggattacctgcacggcgataatagcgatgtgatccccacagacaccatcaagaacacagtgaacgtgctggccaagtttaacggcatcaagtccatcgagacatttgccgtgaccatctgtgagcacttcctgtccagctttaagcacgtgatcagagcccaggtgtacgtggaggaggtgccttggaagaggttcgagaagaatggcgtgaagcacgtgcacgcctttatctacacacccaccggcacccacttctgcgaggtggagcagatcagaaatggccctcccgtgatccactccggcatcaaggacctgaaggtgctgaagaccacccagagcggcttcgagggcttcatcaaggaccagttcaccaccctgcccgaggtgaaggatagatgcttcgccacacaggtgtactgtaagtggagataccaccagggcagagacgtggacttcgaggccacatgggacacagtgagaagcatcgtgctgcagaagtttgccggcccttacgacaagggcgagtacagcccctctgtgcagaagaccctctatgatatccaggtgctctccctgagccgagttcctgagatagaagatatggaaatcagcctgccaaacattcactacttcaatatagacatgtccaaaatgggtctgatcaacaaggaagaggtcttgctgccattagacaatccatatggaaaaattactggtacagtcaagaggaagttgtcttcaagactgtgacggctattatgcgttaccggcgagacgctacggactt

SEQ ID NO: 52 - DNA, 20 bp, synthetic construct
  source 1..20: mol_type = other DNA
ggccagtgaattgtaatacg

SEQ ID NO: 53 - DNA, 20 bp, synthetic construct
  source 1..20: mol_type = other DNA
aactccgtagcgtctcgccg

SEQ ID NO: 54 - DNA, 20 bp, synthetic construct
  source 1..20: mol_type = other DNA
cgccggaaacgcaatagccg

SEQ ID NO: 55 - DNA, 22 bp, synthetic construct
  source 1..22: mol_type = other DNA
cggctattatgcgttaccggcg

SEQ ID NO: 56 - DNA, 60 bp, synthetic construct
  source 1..60: mol_type = other DNA
atgtacaggatgcaactcctgtcttgcattgcactaagtcttgcacttgtcacgaattcg

SEQ ID NO: 57 - DNA, 20 bp, synthetic construct
  source 1..20: mol_type = other DNA
gccaccatgtacaggatgca

SEQ ID NO: 58 - DNA, 20 bp, synthetic construct
  source 1..20: mol_type = other DNA
tcacagtcttgaagacaact

SEQ ID NO: 59 - DNA, 40 bp, synthetic construct
  source 1..40: mol_type = other DNA
agttgtcttcaagactgtgacggctattatgcgttaccgg

SEQ ID NO: 60 - DNA, 40 bp, synthetic construct
  source 1..40: mol_type = other DNA
tgcatcctgtacatggtggctttgctgtattcaacttaac

SEQ ID NO: 61 - DNA, 915 bp, synthetic construct
  source 1..915: mol_type = other DNA
atggcccattatagaaacgattacaagaagaatgatgaggtggagttcgtgaggaccggctatggcaaggacatgatcaaggtgctgcacattcagcgggacggcaagtaccactccatcaaggaggtggctacttccgtccagctgaccctgagcagcaagaaggactatctgcatggggataacagcgatgttatccccacagataccatcaagaacaccgtgaatgtccttgccaagttcaagggcatcaagtccattgagacttttgcagtgacgatctgcgagcacttcctgagtagcttcaagcacgtgatccgcgcccaggtgtatgtggaggaggtgccgtggaagcggttcgagaagaacggcgtgaagcacgtgcacgccttcatatacacccccaccgggactcacttctgcgaggtggagcagatccggaatggaccccccgtgatccactctgggatcaaggacctgaaggtcctgaagaccacccagagtggattcgaggggttcattaaggatcagttcaccaccttgccagaggtgaaggaccggtgcttcgcgacccaggtgtactgcaagtggcggtaccaccagggtcgcgacgtggattttgaggctacttgggacactgttcgcagcatcgtcctgcagaagttcgccggtccttacgacaagggggagtactccccctccgtgcagaagactctgtacgacatccaggtcctcactctcgggcaggtgcccgagattgaggacatggagatctctctgcccaacattcactatctgaatatcgacatgtctaagatgggcctgatcaacaaggaggaggtcctgcttcctcttgacaatccctatgggcggatcacagggactgtcaagaggaagctgacctcccgcttgtga

SEQ ID NO: 62 - DNA, 915 bp, synthetic construct
  source 1..915: mol_type = other DNA
atggcccactatcggaatgactacaagaagaatgacgaggtggagttcgtgaggaccggctatggcaaggacatgatcaaggtgctgcacattcagcgggacggcaagtaccactccatcaaggaggtggctacttccgtccagctgaccctgagcagcaagaaggactatctgcatggggataacagcgatgttatccccacagataccatcaagaacaccgtgaatgtccttgccaagttcaagggcatcaagtccattgagacttttgcagtgacgatctgcgagcacttcctgagtagcttcaagcacgtgatccgcgcccaggtgtacgtggaggaggtgccatggaagcggttcgagaagaacggcgtgaagcatgtgcacgccttcatctacacccccaccgggactcacttctgcgaggtggagcagatccggaatggaccccccgtgatccactctgggatcaaggacctgaaggtcctgaagaccacccagagtggattcgaggggttcattaaggatcagttcaccaccttgccagaggtgaaggaccggtgcttcgcgacccaggtgtactgcaagtggcggtaccaccagggtcgcgacgtggattttgaggctacttgggacacagtgcgcagcatcgtcctgcagaagttcgccggtccttacgacaagggggagtacagcccctccgtgcagaagaccctgtacgatattcaggtgctgaccctggggcaggtgcctgagatcgaggacatggagatcagcctgcccaacattcactacctgaatatcgacatgagcaagatgggtctgatcaacaaggaggaggtgctgctcccccttgacaacccttacggccggatcaccggcaccgtgaagcggaagctgaccagccggctgtga

(19) United States
(12) Patent Application Publication
(10) Pub. No.: US 2024/0228984 A1
(43) Pub. Date: Jul. 11, 2024

(54) CIRCULAR RNAS FOR EXPRESSING URATE OXIDASE, AND PREPARATION METHODS AND USES THEREOF

(71) Applicant: EXCLCIRC (SUZHOU) BIOMEDICAL CO., LTD., Suzhou (CN)

(72) Inventors: Dongsheng DAI, Suzhou (CN); Lynn SHAN, Suzhou (CN); Zilin DAI, Suzhou (CN)

(73) Assignee: EXCLCIRC (SUZHOU) BIOMEDICAL CO., LTD., Suzhou (CN)

(21) Appl. No.: 18/475,347

(22) Filed: Sep. 27, 2023

(30) Foreign Application Priority Data
Jan. 10, 2023 (CN) 202310032709.9

(51) Int. Cl. C12N 9/06; A61P 3/00

(52) U.S. Cl. CPC C12N 9/0048; A61P 3/00; C12Y 107/03003

(57) ABSTRACT

A recombinant nucleic acid molecule for making a circular RNA and a preparation method for the circular RNA are provided. The recombinant nucleic acid molecule comprises elements operably linked to each other and arranged, in a 5′ to 3′ direction, in the following order: (a) an intron fragment which includes a full-length intron; (b) an E2 fragment which includes a downstream exon of the full-length intron; (c) an internal ribosome entry site (IRES) fragment; (d) a urate oxidase coding fragment; and (e) an E1 fragment which includes an upstream exon of the full-length intron; wherein the full-length intron, the downstream exon, and the upstream exon are from a same gene.

### CROSS-REFERENCE TO RELATED APPLICATIONS

This application claims priority to Chinese Patent Application No. 202310032709.9, filed on Jan. 10, 2023, the entire contents of each of which are incorporated herein by reference.

### SEQUENCE LISTING

The instant application contains a Sequence Listing which has been submitted electronically in XML format and is hereby incorporated by reference in its entirety. The XML copy, created on Sep. 25, 2023, is named “209170002US00-sequence listing” and is 64,058 bytes in size.

### TECHNICAL FIELD

The present disclosure belongs to the technical field of molecular biology, and particularly relates to a circular RNA for expressing urate oxidase, as well as preparation methods for the circular RNA and the uses thereof.

### BACKGROUND

Uric acid is a byproduct of a purine metabolism in the human body. When there is a disorder in purine metabolism, it commonly leads to the excessive accumulation of uric acid, resulting in high levels of uric acid in the blood, a condition known as hyperuricemia. Hyperuricemia has emerged as a common health issue with an increasing incidence rate. Prolonged hyperuricemia can lead to the formation of urate calculi, which in turn may trigger gout. Additionally, hyperuricemia is considered a risk factor for cardiovascular and cerebrovascular diseases, chronic nephropathy, and atherosclerosis, all of which pose significant threats to human health. As a result, the treatment of hyperuricemia has garnered significant attention.

Currently, drugs used to treat hyperuricemia primarily consist of xanthine oxidase inhibitors (e.g., allopurinol), as well as uric acid excretion-promoting drugs (e.g., benzbromarone). Drugs such as allopurinol reduce uric acid by inhibiting the activity of xanthine oxidase and blocking the production of uric acid, but they also increase the burden of excreting uric acid precursors in the kidney, and can cause xanthine nephropathy and calculi. Currently, hyperuricemia has been listed as an independent risk factor for cardiovascular diseases. Therefore, it is imperative to find effective treatments.

Urate oxidase, also known as uricase, is an enzyme involved in the metabolic pathway of purine degradation in organisms. During purine metabolism, most organisms produce uric acid, which can be catalyzed by uricase into allantoin. As a result, urate oxidase can be utilized in the treatment of hyperuricemia. However, the currently used urate oxidase is a preparation derived from foreign proteins extracted from the fermentation broth of organisms like Aspergillus niger and Aspergillus flavus. This preparation exhibits strong immunogenicity and can cause allergic reactions such as systemic urticaria-like itching.

Therefore, it is desirable to develop molecules, methods, and system for expressing urate oxidase that exhibits both high activity levels and low immunogenicity for treating hyperuricemia.

### SUMMARY

According to an aspect of the present disclosure, a recombinant nucleic acid molecule for making a circular RNA is provided. The circular RNA is capable of expressing a urate oxidase in cells. The recombinant nucleic acid molecule may include elements operably linked to each other and arranged, in a 5′ to 3′ direction, in the following order:
(a) an intron fragment which includes a full-length intron;
(b) an E2 fragment which includes a downstream exon of the full-length intron;
(c) an internal ribosome entry site (IRES) fragment;
(d) a urate oxidase coding fragment; and
(e) an E1 fragment which includes an upstream exon of the full-length intron;
   wherein the full-length intron, the downstream exon, and the upstream exon are from a same gene.

In some embodiments, an amino acid sequence of the urate oxidase coding fragment has at least 95% similarity with any one of SEQ ID NOs. 1 and 3-8.

In some embodiments, the amino acid sequence of the urate oxidase coding fragment has at least 95% similarity with SEQ ID NO.7, and a corresponding DNA sequence of the urate oxidase coding fragment has at least 95% similarity with SEQ ID NO.9.

In some embodiments, the amino acid sequence of the urate oxidase coding fragment has at least 95% similarity with SEQ ID NO. 8, and a corresponding DNA sequence of the urate oxidase coding fragment has at least 95% similarity with SEQ ID NO. 10.

In some embodiments, a DNA sequence of the urate oxidase coding fragment has at least 95% similarity with SEQ ID NO. 61 or SEQ ID NO. 62.

In some embodiments, the urate oxidase coding fragment includes a pig-derived urate oxidase coding sequence, a baboon-derived urate oxidase coding sequence, or a pig-baboon chimeric sequence.

In some embodiments, the recombinant nucleic acid molecule further includes a signal peptide element, which encodes a signal peptide that is configured to facilitate secreting the urate oxidase outside of the cells, wherein the signal peptide element is positioned between the IRES fragment and the urate oxidase coding fragment.

In some embodiments, the signal peptide includes any one of: an interleukin-2 (IL-2) signal peptide, a human leukocyte antigen (HLA) signal peptide, a leucine-rich α-2 glycoprotein 1 (LRG1) signal peptide, a cholinergic receptor nicotinic alpha 1 subunit (CHRNA1) signal peptide, an apolipoprotein B (APOB) signal peptide, a cystatin D (CST5) signal peptide, a galactosylceramidase (GALC) signal peptide, a gelsolin (GSN) signal peptide, a glycoprotein Ib platelet subunit alpha (GP1BA) signal peptide, a granzyme B (GZMB) signal peptide, a SERPING1 signal peptide, an Interleukin-12 subunit alpha (IL-12A) signal peptide, an interleukin-2 (IL-10) signal peptide, an interleukin 1 receptor-like 1 (IL1RL1) signal peptide, an insulin receptor (INSR) signal peptide, a killer cell Immunoglobulin like receptor, two Ig domains and long cytoplasmic tail 1 (KIR2DL1) signal peptide, a kallikrein related peptidase 14 (KLK14) signal peptide, a lacritin (LACRT) signal peptide, and a lymphocyte activation gene-3 (LAG3) signal peptide.

In some embodiments, an amino acid sequence of the signal peptide has at least 95% similarity with any one of SEQ ID NOs. 11-29.

In some embodiments, the recombinant nucleic acid molecule further includes a signal positioning element, which encodes a signal positioning peptide that is configured for positioning the urate oxidase to a peroxisome, wherein the signal positioning element is positioned between the IRES fragment and the urate oxidase coding fragment.

In some embodiments, an amino acid sequence of the signal positioning peptide has at least 97% or 100% similarity with SRL.

In some embodiments, the IRES element is derived from a Taura syndrome virus, Triatoma virus, Theiler's murine encephalomyelitis virus, simian virus 40, Solenopsis invicta virus 1, Rhopalosiphum padi virus, reticuloendotheliosis virus, Poliovirus type 1, Plautia stali intestine virus, Kashmir bee virus, human rhinovirus 2, human immunodeficiency virus type 1, Homalodisca coagulata virus-1, Himetobi P virus, hepatitis C virus, hepatitis A virus, hepatitis B virus, foot-and-mouth disease virus, human enterovirus 71, equine rhinovirus, Ectropis obliqua picorna-like virus, Encephalomyocarditis virus (EMCV), Drosophila C virus, tobacco mosaic virus, cricket paralysis virus, bovine viral diarrhea virus 1, black queen cell virus, aphid lethal paralysis virus, avian encephalomyclitis virus, acute bee paralysis virus, Hibiscus Chlorotic Ringspot virus, hog cholera virus, salivary virus, Coxsackie virus, Parechovirus, simian picornavirus, turnip crinkle virus, Coxsackie virus B1 (CVB1), Coxsackie virus B2 (CVB2), or Coxsackie virus B3 (CVB3).

In some embodiments, the IRES element is derived from the CVB3.

In some embodiments, the IRES element is cloned from a gene coding a protein selected from a group consisting of: human FGF2, human SFTPA1, human AMLI/RUNXI, Drosophila antenna, human AQP4, human AT1R, human BAG-1, human BCL2, human BiP, human cIAP-1, human c-myc, human eIF4G, mouse NDST4L, human LEF1, mouse HIF1α, human n-myc, mouse Gtx, human p27KipI, human PDGF2/c-sis, human p53, human Pim-1, mouse Rbm3, Drosophila reaper, dog Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, and an aptamer of eIF4G.

In some embodiments, the IRES element includes ribosome recognition sequences pIRES1-pIRES10.

In some embodiments, the IRES element is pIRES9.

In some embodiments, a DNA sequence of the IRES element has at least 95% similarity with any one of SEQ ID NOs. 30-40.

In some embodiments, a DNA sequence of the IRES element has at least 95% similarity with SEQ ID NO. 38 or SEQ ID NO. 40.

In some embodiments, the intron fragment includes an intron of the pre-tRNA_{Leu} gene of genus Anabaena; the E2 fragment includes a downstream exon of the intron of the pre-tRNA_{Leu} gene of genus Anabaena; and the E1 fragment includes an upstream exon of the intron of the pre-tRNA_{Leu} gene of genus Anabaena.

In some embodiments, a nucleotide sequence of the intron fragment has at least 95% similarity with SEQ ID NO. 41, a nucleotide sequence of the E2 fragment has at least 95% similarity with any one of SEQ ID NO. 42 to SEQ ID NO. 45, AAAATCCG, AAAATC, AAAA, and AA, a nucleotide sequence of the E1 fragment has at least 95% similarity with any one sequence of SEQ ID NO. 46 to SEQ ID NO. 49, GGACTT, ACTT, TT, and CTT.

In some embodiments, the recombinant nucleic acid molecule further includes a 5′ homology arm sequence and a 3′ homology arm sequence positioned between the E2 fragment and the E1 fragment.

In some embodiments, the intron fragment is further preceded by a promoter which initiates in vitro transcription of the recombinant nucleic acid molecule.

In some embodiments, the promoter is one of a T7 promoter, a T3 promoter, and an SP6 promoter.

In some embodiments, the recombinant nucleic acid molecule is a vector.

According to another aspect of the present disclosure, a linear RNA which is produced based on the recombinant nucleic acid molecule is provided.

According to another aspect of the present disclosure, a circular RNA which is produced based on the recombinant nucleic acid molecule is provided.

According to another aspect of the present disclosure, a method for preparing a circular RNA based on the recombinant nucleic acid molecule is provided. The method may include: obtaining a linear RNA by performing an in vitro transcription reaction on the recombinant nucleic acid molecule; and allowing the linear RNA to self-circularize to produce the circular RNA.

In some embodiments, the recombinant nucleic acid molecule is generated by in vitro synthesis.

In some embodiments, the recombinant nucleic acid molecule is generated by: constructing a recombinant plasmid that includes a promoter and a sequence of the nucleic acid molecule; obtaining the nucleic acid molecule by PCR amplifications with the recombinant plasmid as a template, using a forward primer at the beginning of the promoter and a reverse primer at the end of the E1 fragment.

In some embodiments, the promoter is one of a T7 promoter, a T3 promoter, and an SP6 promoter.

In some embodiments, the promoter is the T7 promoter; and sequences of the forward primer and the reverse primer have at least 95% similarity with SEQ ID NO. 52 and SEQ ID NO. 53, respectively.

In some embodiments, the nucleic acid molecule is generated by: constructing a recombinant plasmid that includes a sequence of the nucleic acid molecule; and digesting the recombinant plasmid with a type IIS restriction endonuclease or a type II blunt restriction endonuclease to obtain the nucleic acid molecule.

In some embodiments, the type IIS restriction endonuclease comprises at least one of Acu I, Alw I, Bae I, Bbs I, BbV I, Bcc I, BceA I, Bcg I, BciV I, Bmr I, Bpm I, BpuE I, BsaX I, BseR I, Bsg I, BsmA I, BsmBI-v2, BsmF1, Bsm I, BspCN I, BspM I, BspQ I, BsrD I, Bsr I, BtgZ I, BtsC I, BtsI-v2, BtsImut I, CspC I, Ear I, Eci I, Esp3 I, Fau I, Fok I, Hga I, Hph I, HpyA V, Mbo II, Mly I, Mme I, Mnl I, NmeA III, PaqC I, Ple I, Sap I, and SfaN I.

In some embodiments, the type II blunt restriction endonuclease comprises at least one of Afe I, Alu I, BsaA I, BstU I, BstZ17 I, Dra I, EcoR V, Fsp I, Hae III, Hpa I, Hinc II, Msc I, MspA1 I, Nae I, Nru I, Pme I, Pm II, Pvu II, Rsa I, Sca I, Sfo I, Sma I, SnaB I, Ssp I, Stu I, or Swa I.

In some embodiments, the method further includes encapsulating the circular RNA by lipid nanoparticles (LNP).

In some embodiments, the encapsulating the circular RNA by LNP further includes: dissolving the LNP into ethyl alcohol to obtain a LNP solution; dissolving the circular RNA into a sodium acetate solution to obtain a circular RNA solution; and obtaining the LNP-encapsulated circular RNA by making the LNP solution and the circular RNA solution pass through a microfluidic device.

In some embodiments, the LNP includes SM102, PEG2000, DSPC, and cholesterol, and a molar ratio of SM102:DSPC:cholesterol:DME-PEG2000 is 30-60:3-20:25-50:0.2-5.

In some embodiments, a molar N/P ratio of the LNP solution to the circular RNA solution is 2-8:1.

According to another aspect of the present disclosure, a method for reducing a uric acid in a subject is provided. The method includes administering the circular RNA, in a pharmaceutically acceptable amount, to the subject.

According to another aspect of the present disclosure, a method for treating a disease with a high uric acid level in a subject is provided. The method includes administering the circular RNA according to claim 35, in a pharmaceutically acceptable amount, to the subject.

In some embodiments, the disease includes hyperuricemia.

According to another aspect of the present disclosure, a use of the circular RNA in the preparation of a drug for reducing a uric acid or treating a disease with a high uric acid level in a subject.

### BRIEF DESCRIPTION OF THE DRAWINGS

The present disclosure is further illustrated in terms of exemplary embodiments, and these exemplary embodiments are described in detail with reference to the drawings. These embodiments are not limited, wherein:

FIG. 1 is a schematic diagram illustrating an exemplary recombinant nucleic acid molecule named as CVB3-IL2-PBC in Example 1;

FIG. 2 is a schematic diagram illustrating an exemplary recombinant nucleic acid molecule named as IRES9-IL2-PBC in Example 1;

FIG. 3 shows a purification result of circular RNA in Example 1;

FIG. 4 shows an animal experiment scheme in Example 2;

FIG. 5 shows urate oxidase activity in a mouse liver of each group;

FIG. 6 is a histogram illustrating the content of serum uric acid in a control group;

FIG. 7 is a histogram illustrating the content of serum uric acid in a hyperuricemia group;

FIG. 8 is a histogram illustrating the content of serum uric acid in a group administrated with a high dose of CVB3;

FIG. 9 is a histogram illustrating the content of serum uric acid in a group administrated with a low dose of CVB3; and

FIG. 10 is a histogram illustrating the content of serum uric acid in a group administrated with a high dose of IRES9;

FIG. 11 is a histogram illustrating the content of serum uric acid in a group administrated with a low dose of IRES9;

FIG. 12 is a is a histogram illustrating the content of serum uric acid in a group administrated with benzbromarone;

FIG. 13 is a histogram illustrating the content of serum uric acid in a group administrated with urate oxidase;

FIG. 14 shows anti-urate oxidase antibody titer of each group at 10-fold dilution of serum samples; and

FIG. 15 shows anti-urate oxidase antibody titer of each group at 100-fold dilution of serum samples.

### DETAILED DESCRIPTION

The following clearly and completely describes the technical solutions of the present disclosure with reference to the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

As shown in the present disclosure and claims, unless the context clearly indicates exceptions, the words “a,” “an,” “one,” and/or “the” do not specifically refer to the singular form, but may also include the plural form. The terms “including” and “comprising” or the like only suggest that the steps and elements that have been clearly identified are included, and these steps and elements do not constitute an exclusive list, and the method or device may also include other steps or elements.

The flowcharts used in the present disclosure may illustrate operations executed by the system according to embodiments in the present disclosure. It should be understood that a previous operation or a subsequent operation of the flowcharts may not be accurately implemented in order. Conversely, various operations may be performed in inverted order, or simultaneously. Moreover, other operations may be added to the flowcharts, and one or more operations may be removed from the flowcharts.

### Definition

As used herein, the “intron” refers to a non-coding fragment in a DNA sequence. The “exon” refers to a coding fragment in a DNA sequence, which can be transcribed and translated into a portion of the protein. The DNA sequence of a gene may include an intron and an exon. In a process of transcription, the gene is transcribed into an intermediate molecule, which is referred to as pre-messenger RNA (or linear RNA). In the pre-messenger RNA, an intron is transcribed, but it is not retained in a mature mRNA.

As used herein, “splicing” refers to a process that an intron is removed from the pre-messenger RNA, and an exon is connected to form a mature mRNA molecule. Splicing plays a significant role in regulating gene expression. The way and selectivity of the splicing may lead to various combinations of exons, leading to the production of multiple distinct mature mRNAs. Consequently, this process has an impact on the composition of proteins during transcription and translation.

As used herein, the “full-length intron” refers to a complete intron sequence extending from a starting boundary of an exon to an ending boundary of a next exon in a DNA sequence of a gene.

Although the intron does not directly encode proteins, the intron may play an important role in gene expression regulation, evolution, etc. By regulating and splicing, cells produce diverse proteins, thus adapting to different biological processes and environmental conditions.

As used herein, the “downstream exon” refers to an exon following an intron in a sequence of the pre-messenger RNA corresponding to a DNA sequence of a gene. The upstream exon is usually an exon before the downstream exon. The “upstream” and “downstream” are used herein to represent a spatial position of elements in a genome or an RNA sequence. For example, “upstream” refers to a direction farther away from an intron, while “downstream” refers to a direction closer to the intron.

As used herein, “transcription” refers to a process of synthesizing RNA using a DNA molecule as a template. Inside cellular structures, DNA carries the encoded biological genetic information. To effectively execute the biological genetic information in the cells, it is necessary to replicate the biological genetic information in the DNA into RNA molecules. This replication enables the production of proteins or the accomplishment of other functions during the translation process.

According to some embodiments of the present disclosure, provided is a recombinant nucleic acid molecule for making a circular RNA.

Circular ribonucleic acids (circular RNA, or circRNAs) are an important class of the regulatory non-coding RNA. A circular RNA usually includes an enclosed circular structure, and is generally not affected by RNA exonucleases. circular RNAs are often stable in nature and can regulate gene expression through a variety of mechanisms. Circular RNAs have promise as therapeutic agents. In the present disclosure, the circular RNA is capable of expressing a urate oxidase in cells, and thus can be configured to reduce uric acid level.

The recombinant nucleic acid molecule may include elements operably linked to each other and arranged, in a 5′ to 3′ direction, in the following order:
(a) an intron fragment which includes a full-length intron;
(b) an E2 fragment which includes a downstream exon of the full-length intron;
(c) an internal ribosome entry site (IRES) fragment;
(d) a urate oxidase coding fragment; and
(e) an E1 fragment which includes an upstream exon of the full-length intron; wherein the full-length intron, the downstream exon, and the upstream exon are from a same gene.

The urate oxidase coding fragment refers to a coding region (or coding sequence) of a urate oxidase gene that is translated to form urate oxidase. The urate oxidase gene may be derived from animals, plants, microorganism, etc. In some embodiments, the urate oxidase gene may be derived from a pig (Sus scrofa, or porcine), a sheep, a horse, a cow (Bos taurus), a baboon (Papio Anubis), a dog (Canis lupus familiaris), etc. In some embodiments, the urate oxidase coding fragment may be from a chimeric sequence, such as a pig-baboon chimeric (also referred to as porcine-baboon chimera, PBC) sequence, a pig-horse chimeric sequence, etc. The chimeric sequence here refers to a sequence which is combined from two or more urate oxidase gene sequences. In some embodiments, the urate oxidase coding fragment may include a pig-baboon chimeric sequence, which means the urate oxidase coding fragment consists of a pig-derived sequence and a baboon-derived sequence. Merely by way of illustration, 1th-225th amino acids of an amino acid sequence of a pig-derived urate oxidase and 226th-304th amino acids of an amino acid sequence of a baboon-derived urate oxidase were combined to form pig-baboon chimeric amino acid sequence.

In some embodiments, an amino acid sequence of the urate oxidase coding fragment has at least 95%, 96%, 97%, 98%, or 99% similarity with any one of SEQ ID NOs. 1 and 3-8. In some embodiments, the amino acid sequence of the urate oxidase coding fragment has 100% similarity with any one of SEQ ID NOs. 1 and 3-8.

In some embodiments, the amino acid sequence of the urate oxidase coding fragment has at least 95%, 96%, 97%, 98%, or 99% similarity with SEQ ID NO.7, and a corresponding DNA sequence of the urate oxidase coding fragment has at least 95%, 96%, 97%, 98%, or 99% similarity with SEQ ID NO.9. In some embodiments, the amino acid sequence of the urate oxidase coding fragment has 100% similarity with SEQ ID NO.7, and a corresponding DNA sequence of the urate oxidase coding fragment has 100% similarity with SEQ ID NO.9.

In some embodiments, the amino acid sequence of the urate oxidase coding fragment has at least 95%, 96%, 97%, 98%, or 99% similarity with SEQ ID NO. 8, and the corresponding DNA sequence of the urate oxidase coding fragment has at least 95%, 96%, 97%, 98%, or 99% similarity with SEQ ID NO. 10. In some embodiments, the amino acid sequence of the urate oxidase coding fragment has 100% similarity with SEQ ID NO. 8, and the corresponding DNA sequence of the urate oxidase coding fragment has 100% similarity with SEQ ID NO. 10. In some embodiments, the urate oxidase coding fragment also includes termination codon. For example, SEQ ID NO.9 includes termination codon.

In some embodiments, the DNA sequence of the urate oxidase coding fragment has at least 95%, 96%, 97%, 98%, or 99% similarity with SEQ ID NO. 61 or SEQ ID NO. 62. In some embodiments, the DNA sequence of the urate oxidase coding fragment has 100% similarity with SEQ ID NO. 61 or SEQ ID NO. 62.

Additionally or alternatively, the recombinant nucleic acid molecule further includes a signal peptide element, which encodes a signal peptide that is configured to facilitate secreting the urate oxidase outside of the cells. The signal peptide element is positioned between the IRES fragment and the urate oxidase coding fragment.

In some embodiments, the signal peptide includes an interleukin-2 (IL-2) signal peptide, a human leukocyte antigen (HLA) signal peptide, a leucine-rich α-2 glycoprotein 1 (LRG1) signal peptide, a cholinergic receptor nicotinic alpha 1 subunit (CHRNA1) signal peptide, an apolipoprotein B (APOB) signal peptide, a cystatin D (CST5) signal peptide, a galactosylceramidase (GALC) signal peptide, a gelsolin (GSN) signal peptide, a glycoprotein Ib platelet subunit alpha (GP1BA) signal peptide, a granzyme B (GZMB) signal peptide, a SERPING1 signal peptide, an Interleukin-12 subunit alpha (IL-12A) signal peptide, an interleukin-2 (IL-10) signal peptide, an interleukin 1 receptor-like 1 (IL1RL1) signal peptide, an insulin receptor (INSR) signal peptide, a killer cell Immunoglobulin like receptor, two Ig domains and long cytoplasmic tail 1 (KIR2DL1) signal peptide, a kallikrein related peptidase 14 (KLK14) signal peptide, a lacritin (LACRT) signal peptide, and a lymphocyte activation gene-3 (LAG3) signal peptide, or the like, or any combination thereof.

In some embodiments, an amino acid sequence of the signal peptide has at least 95%, 96%, 97%, 98%, or 99% similarity with any one of SEQ ID NOs. 11-29. In some embodiments, the amino acid sequence of the signal peptide has 100% similarity with any one of SEQ ID NOs. 11-29.

Additionally or alternatively, the recombinant nucleic acid molecule further includes a signal positioning element, which encodes a signal positioning peptide that is configured for positioning the urate oxidase to a peroxisome, where there are a lot of digestive enzymes for breaking down toxic materials in the cell and oxidative enzymes for metabolic activity. The signal positioning element is positioned between the IRES fragment and the urate oxidase coding fragment.

In some embodiments, an amino acid sequence of the signal positioning peptide has at least 97%, 98%, or 99% similarity with SRL. In some embodiments, the amino acid sequence of the signal positioning peptide has 100% similarity with SRL. In some embodiments, a corresponding DNA sequence of the signal positioning peptide has at least 95%, 96%, 97%, 98%, or 99% similarity with TCAAGACTG. In some embodiments, a corresponding DNA sequence of the signal positioning peptide has 100% similarity with TCAAGACTG. Additionally or alternatively, some amino acid sequences (e.g., SEQ ID NO. 2) of the urate oxidase coding fragment includes the amino acid sequence of the signal positioning peptide (e.g., SRL).

The IRES element may be transcribed to an RNA molecule that is capable of recruiting ribosomes for a translation reaction to obtain the target peptide. The IRES element may be derived from a virus which includes a Taura syndrome virus, Triatoma virus, Theiler's murine encephalomyelitis virus, simian virus 40, Solenopsis invicta virus 1, Rhopalosiphum padi virus, reticuloendotheliosis virus, Poliovirus type 1, Plautia stali intestine virus, Kashmir bee virus, human rhinovirus 2, human immunodeficiency virus type 1, Homalodisca coagulata virus-1, Himetobi P virus, hepatitis C virus, hepatitis A virus, hepatitis B virus, foot-and-mouth disease virus, human enterovirus 71, equine rhinovirus, Ectropis obliqua picorna-like virus, Encephalomyocarditis virus (EMCV), Drosophila C virus, tobacco mosaic virus, cricket paralysis virus, bovine viral diarrhea virus 1, black queen cell virus, aphid lethal paralysis virus, avian encephalomyclitis virus, acute bee paralysis virus, Hibiscus Chlorotic Ringspot virus, hog cholera virus, salivary virus, Coxsackie virus, Parechovirus, simian picornavirus, turnip crinkle virus, Coxsackie virus B1 (CVB1), Coxsackie virus B2 (CVB2), or Coxsackie virus B3 (CVB3).

In some embodiments, the IRES element may be derived from the CVB3, the IRES element is cloned from a gene coding a protein selected from a group consisting of: human FGF2, human SFTPA1, human AMLI/RUNXI, human AQP4, human AT1R, human BAG-1, human BCL2, human BiP, human cIAP-1, human c-myc, human eIF4G, mouse NDST4L, human LEF1, mouse HIF1α, human n-myc, mouse Gtx, human p27KipI, human PDGF2/c-sis, human p53, human Pim-1, mouse Rbm3, Drosophila reaper, dog Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, and an aptamer of eIF4G.

In some embodiments, the IRES element includes ribosome recognition sequences pIRES1-pIRES10. In some embodiments, the IRES element may be derived from human cells which are predicted and verified based on polyribosome profiling data. In some embodiments, the IRES element is pIRES9.

In some embodiments, a DNA sequence of the IRES element has at least 95%, 96%, 97%, 98%, or 99% similarity with any one of SEQ ID NOs. 30-40. In some embodiments, the DNA sequence of the IRES element has at least 95%, 96%, 97%, 98%, or 99% similarity with SEQ ID NO. 38 or SEQ ID NO. 40. In some embodiments, the DNA sequence of the IRES element is any one of SEQ ID NOs. 30-40.

In some embodiments, the intron fragment includes an intron of the pre-tRNA_{Leu} gene of genus Anabaena; the E2 fragment includes a downstream exon of the intron of the pre-tRNA_{Leu} gene of genus Anabaena; and the E1 fragment includes an upstream exon of the intron of the pre-tRNA_{Leu} gene of genus Anabaena.

In some embodiments, a nucleotide sequence of the intron fragment may have at least 95% similarity with SEQ ID NO. 41. In some embodiments, a nucleotide sequence of the E2 fragment may have at least 95% similarity with any one of SEQ ID NO. 42 to SEQ ID NO. 45, AAAATCCG, AAAATC, AAAA, and AA. In some embodiments, a nucleotide sequence of the E1 fragment may have at least 95% similarity with any one sequence of SEQ ID NO. 46 to SEQ ID NO. 49, GGACTT, ACTT, TT, and CTT. In some embodiments, the nucleotide sequence of the intron fragment may have 100% similarity with SEQ ID NO. 41. In some embodiments, the nucleotide sequence of the E2 fragment may have 100% similarity with any one of SEQ ID NO. 42 to SEQ ID NO. 45, AAAATCCG, AAAATC, AAAA, and AA. In some embodiments, the nucleotide sequence of the E1 fragment may have 100% similarity with any one sequence of SEQ ID NO. 46 to SEQ ID NO. 49, GGACTT, ACTT, TT, and CTT.

Additionally or alternatively, the recombinant nucleic acid molecule may further include a 5′ homology arm sequence and a 3′ homology arm sequence positioned between the E2 fragment and the E1 fragment. The 5′ homology arm sequence may be usually positioned at the 5′ end of the DNA molecule; the 3′ homology arm sequence may be usually positioned at the 3′ end of the DNA molecule. The 5′ homology arm sequence and the 3′ homology arm sequence are designed and inserted in the recombinant nucleic acid molecule for homologous recombination.

In some embodiments, the intron fragment is further preceded by a promoter which initiates in vitro transcription of the recombinant nucleic acid molecule.

In some embodiments, the promoter may be a T7 promoter, a T3 promoter, an SP6 promoter, or the like, or any combination thereof.

In some embodiments, the recombinant nucleic acid molecule may be a vector. As used herein, the vector refers to a tool used to carry an exogenous DNA fragment and undergo a transcription reaction in a cell to produce an RNA.

The vector is usually a circular DNA molecule, such as a plasmid or a virus (e.g., adenovirus, Adeno-associated virus, etc.), bacterial artificial chromosome (BAC), yeast artificial chromosome (YAC), etc. These vectors have the ability to self-replicate and may replicate independently in cells, while also may carry exogenous genes such as protein coding genes and RNA genes, or the like.

In some embodiments, the vector may be designed to include a specific promoter, a regulatory element, and a terminator to allow the exogenous DNA within the cell to transcribe and produce the RNA. These RNA molecules may be mRNAs for encoding proteins or other non-coding RNAs.

In some embodiments, an in vitro transcription template may be obtained based on the above-mentioned vector, and the circular RNA may be formed in the in vitro transcription reaction based on the in vitro transcription template.

The in vitro transcription template may be obtained through various methods. For example, the in vitro transcription template may be directly obtained through an artificial in vitro synthesis. In some embodiments, the in vitro transcription template may be obtained by constructing plasmids for PCR amplification, or by cutting plasmids with a restriction endonuclease.

The obtained circular RNA expressing the recombinant urate oxidase can significantly increase the expression level of the urate oxidase, can reduce the uric acid level effectively, exhibit low immunogenicity and have good safety. The circular RNA capable of continuously expressing the urate oxidase ensures long-term effectiveness, reducing the need for frequent medication. A single injection of the circular RNA can maintain low uric acid levels for up to 28 days. Consequently, this method offers a straightforward procedure and high efficiency in promoting cyclization.

According to some embodiments of the present disclosure, provided is a method for preparing a circular RNA based on the recombinant nucleic acid molecule described above. The method may include obtaining a linear RNA by performing an in vitro transcription reaction on the recombinant nucleic acid molecule; and allowing the linear RNA to self-circularize to produce the circular RNA.

In some embodiments, the recombinant nucleic acid molecule is generated by in vitro synthesis.

In some embodiments, the recombinant nucleic acid molecule may be generated by: constructing a recombinant plasmid that includes a promoter and a sequence of the nucleic acid molecule; and obtaining the nucleic acid molecule by PCR amplifications with the recombinant plasmid as a template, using a forward primer and a reverse primer at the end of the E1 fragment.

In some embodiments, the promoter may be a T7 promoter, a T3 promoter, an SP6 promoter, or the like, or any combination thereof. In some embodiments, the promoter may be the T7 promoter; and sequences of the forward primer and the reverse primer have at least 95% similarity with SEQ ID NO. 52 and SEQ ID NO. 53, respectively.

In some embodiments, the recombinant nucleic acid molecule may be generated by: constructing a recombinant plasmid that includes a sequence of the nucleic acid molecule; digesting the recombinant plasmid with a type IIS restriction endonuclease or a type II blunt restriction endonuclease to obtain the nucleic acid molecule.

The type IIS restriction endonuclease may include at least one of Acu I, Alw I, Bae I, Bbs I, BbV I, Bcc I, BceA I, Bcg I, BciV I, Bmr I, Bpm I, BpuE I, BsaX I, BseR I, Bsg I, BsmA I, BsmBI-v2, BsmF1, Bsm I, BspCN I, BspM I, BspQ I, BsrD I, Bsr I, BtgZ I, BtsC I, BtsI-v2, BtsImut I, CspC I, Ear I, Eci I, Esp3 I, Fau I, Fok I, Hga I, Hph I, HpyA V, Mbo II, Mly I, Mme I, Mnl I, NmeA III, PaqC I, Ple I, Sap I, and SfaN I.

The type II blunt restriction endonuclease may include at least one of Afe I, Alu I, BsaA I, BstU I, BstZ17 I, Dra I, EcoR V, Fsp I, Hae Ill, Hpa I, Hinc II, Msc I, MspA1 I, Nae I, Nru I, Pme I, Pm II, Pvu II, Rsa I, Sca I, Sfo I, Sma I, SnaB I, Ssp I, Stu I, or Swa I.

In some embodiments, the method further includes encapsulating the circular RNA by lipid nanoparticles (LNP). LNP encapsulation can enhance the advantage of circular RNA in protein production. LNPs are the most advanced nanoparticle carriers that can be used to target specific cells using endogenous or exogenous ligands by encapsulating the circular RNA. Endocytosis of LNPs destabilizes the endosomal membrane and releases the circular RNA into the cytoplasm. LNPs can solve many of the problems with circular RNA molecules, making them less susceptible to degradation and promoting cellular uptake.

In some embodiments, the encapsulating the circular RNA by LNP further includes: dissolving the LNP into ethyl alcohol to obtain a LNP solution; dissolving the circular RNA into a sodium acetate solution to obtain a circular RNA solution; and obtaining the LNP-encapsulated circular RNA by making the LNP solution and the circular RNA solution pass through a microfluidic device.

The LNP may include SM102, DME-PEG2000, DSPC, and cholesterol. In some embodiments, a molar ratio of SM102:DSPC:cholesterol:DME-PEG2000 is in a range of 30-60:3-20:25-50:0.2-5, or in a range of 35-58:5-18:28-45:0.5-3, or in a range of 40-55:8-15:30-43:0.8-2, etc. In some embodiments, the molar ratio of SM102:DSPC:cholesterol:DME-PEG2000 is 50:10:38.5:1.5.

In some embodiments, a molar N/P ratio of the LNP solution to the circular RNA solution is in a range of 2-8:1, or 2-7:1, or 2-6:1, or 2-5:1, etc. In some embodiments, the molar N/P ratio of the LNP solution to the circular RNA solution is 3:1, or 2:1, or 4:1, or 5:1, or 6:1, or 7:1, or 8:1. As used herein, the molar N/P ratio (also referred to as N/P ratio, N:P ratio, or NP) is defined as a ratio of amine groups in the ionizable lipid of the LNP solution to the phosphate groups on the circRNA backbone.

It should be noted that in addition to LNP, other circular RNA-based drug delivery systems can be used, e.g., gold nanoparticles (AuNPs), engineered exosomes, which are not intended to be limiting.

According to some embodiments of the present disclosure, provided is a method for reducing a uric acid in a subject. The method may include administering the circular RNA, in a pharmaceutically acceptable amount, to the subject.

According to some embodiments of the present disclosure, provided is a method for treating a disease with a high uric acid level in a subject. The method includes administering the circular RNA, in a pharmaceutically acceptable amount, to the subject.

As used herein, “subject” refers to a human or animal. Usually, the animal is a vertebrate such as a primate (e.g., chimpanzees, cynomolgus monkeys, spider monkeys, and macaques), rodent (e.g., mice, rats, woodchucks, ferrets, rabbits and hamsters), domestic animal or game animal (e.g., cows, horses, pigs, deer, bison, buffalo, feline species). In some embodiments, the subject is a mammal, e.g., a primate, e.g., a human.

As used herein, the term “pharmaceutically acceptable amount” refers to an amount of the circular RNA that provides a therapeutic benefit in the treatment of the disease with a high uric acid level or the reduction of uric acid, e.g., an amount that provides a statistically significant decrease in, e.g., serum uric acid. Determination of a pharmaceutically acceptable amount is well within the capability of those skilled in the art. Generally, a pharmaceutically acceptable amount can vary with the subject's history, age, condition, sex, as well as the severity and type of the medical condition in the subject, and administration of other pharmaceutically active agents.

In some embodiments, the circular RNA may be administered to the subject at a dose of 0.1 ug/kg to 200 ug/kg, 0.1 ug/kg to 150 ug/kg, 0.1 ug/kg to 100 ug/kg, 1 ug/kg to 150 ug/kg, etc. In some embodiments, the circular RNA is administered every two days, every four days, weekly, bi-weekly, or at any interval within 3 years.

As used herein, the term “disease with a high uric acid level” refers to a disease, disorder or medical condition which can cause the high uric acid level or generated due to the high uric acid level directly or indirectly. Exemplary diseases include hyperuricemia, urate calculi, gout, cardiovascular and cerebrovascular diseases, chronic nephropathy, atherosclerosis, or the like, or any combination thereof.

The disclosure is illustrated by the following examples, which are not intended to be limiting.

### EXAMPLES

### Reagents

Hypoxanthine (Macklin, H811076/68-94-0); ethambutol (Macklin, E877558/74-55-5); benzbromarone (Aladdin, B131634/3562-84-3); urate oxidase (Macklin, U833293/9002-12-4); carboxymethyl cellulose (Macklin, C804618/9004-32-4); urate oxidase activity detection kit (Solarbio Bioscience & Technology Co., LTD, BC4435); uric acid content detection kit (abbkine, KTB1510); 0.9% sodium chloride in normal saline (Klus Pharma); 4% paraformaldehyde (Solarbio Bioscience & Technology Co., LTD, P1110).

### Preparation of Solutions

A hypoxanthine suspension (100 mg/ml) is prepared by adding 1.5 g of hypoxanthine (gavage, 1000 mg/kg) into 15 ml of ddH₂O. A ethambutol solution (25 mg/ml) is prepared by adding 250 mg of ethambutol (intraperitoneal injection, 250 mg/kg) into 10 ml of ddH₂O. A benzbromarone suspension (30 mg/ml) is prepared by adding 75 mg of benzbromarone (gavage, 30 mg/kg) into 2.5 ml of a 1% CMC buffer (100 mg of carboxymethyl cellulose dissolved in 10 ml of ddH₂O). 10 mg of the urate oxidase (tail vein injection, 10 mg/kg) is added into 1 ml of ddH₂O, and then subpackaging into 200 μl×5 pieces, and 1800 μl of ddH₂O is added into the 200 μl (10 mg/ml) each time in use, to obtain a working concentration 1 mg/ml of a urate oxidase solution.

### Instruments

A 1 ml syringe with a needle (Ming An medical instrument); a 2.5 ml syringe with a needle (Ming an medical instrument); an U40 syringe (Braun, Germany); a vortex instrument (Mobio, Vortex 1311); a water bath kettle (SENCO, W5-100SP); a gavage needle for mice (BOLIGE, #8); a Microplate Reader (BIO-RAD, 111-7); a clean bench (Sujing Group, China, SW-CJ-2E); an ultra-pure water instrument (Millipore, ZRQSVP300); a 96-well high adsorption ELISA plate (Jet Bio, FEP-101-896); a magnetic stirrer (Shanghai Meiyingpu Instrument, 08-3G); and a THZ-C thermostatic oscillator (Jiangsu Taicang Laboratorial Equipment Factory, B0101123).

### Laboratory Animals

48 male C57 mice of (20±2) g, which are provided by the Laboratory Animal Center, Fourth Military Medical University, and fed according to the requirements of specification; under the animal license number: SCXK (Shan) 2019-001.

### Example 1 Preparation of Circular RNA for Expressing Urate Oxidase

In this example, taking a recombinant urate oxidase (the amino acid sequence is shown in SEQ ID NO.7) from a pig urate oxidase and a baboon urate oxidase as an example, a recombinant nucleic acid molecule for making a circular RNA which can express urate oxidase were synthesized, which were named as CVB3-IL2-PBC (the DNA sequence is shown in SEQ ID NO.50) and IRES9-IL2-PBC (the DNA sequence is shown in SEQ ID NO.51) respectively. The recombinant urate oxidase also referred to as pig-baboon chimera (PBC).

### 1. Synthesis of PBC Sequence

A sequence of a PBC was synthesized by General Biosystems (Anhui) Co., Ltd. The sequence of the PBC, i.e., the recombinant urate oxidase (the amino acid sequence is shown in SEQ ID NO.7), was formed by combining the 1th-225th amino acids of the amino acid sequence of the pig urate oxidase (the amino acid sequence is shown in SEQ ID NO.4) and the 226th-304th amino acids of the amino acid sequence of the baboon urate oxidase (the amino acid sequence is shown in SEQ ID NO.3).

### 2. Preparation of Plasmid Capable of Expressing PBC.

In this example, an intron (intron fragment), an E2 and an E1 were designed from pre-tRNA_{Leu} gene of genus Anabaena, which are not intended to be limiting. These fragments were connected together and inserted between the 5′ homology arm sequence (the DNA sequence is shown in SEQ ID NO.54) and the 3′ homology arm sequence (the DNA sequence is shown in SEQ ID NO.55) of the vector through homologous recombination, and the recombinant vector was transformed into competent cells and screened under stress to obtain a recombinant plasmid capable of expressing PBC.

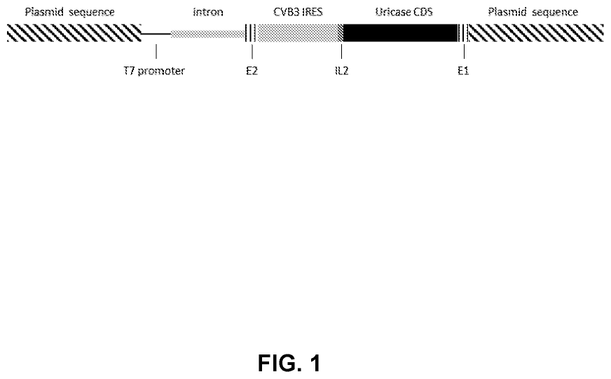

A schematic diagram of the construction of the CVB3-IL2-PBC was as shown in FIG. 1. The CVB3-IL2-PBC includes an Intron (the DNA sequence is shown in SEQ ID NO.41); an E2 (as shown in SEQ ID NO.42 used in this example); a 5′ homology arm sequence (the DNA sequence is shown in SEQ ID NO.54); an IRES sequence (the DNA sequence is shown in SEQ ID NO.40); a gene sequence of IL-2 signal peptide (the DNA sequence is shown in SEQ ID NO.56); a urate oxidase coding fragment (the amino acid is shown in SEQ ID NO.8); a 3′ homology arm sequence (the DNA sequence is shown in SEQ ID NO.55); an E1 (the DNA sequence is shown in SEQ ID NO.46 in this example).

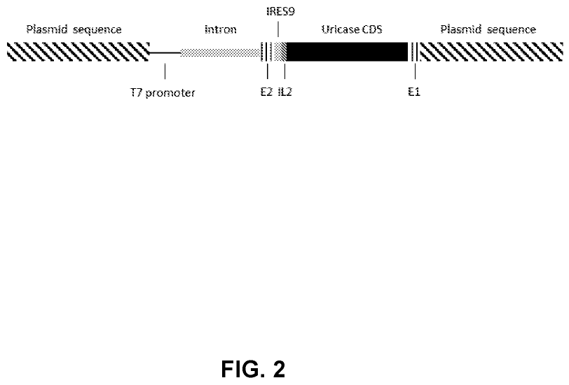

A schematic diagram of the construction of the IRES9-IL2-PBC was as shown in FIG. 2. The IRES9-IL2-PBC includes an Intron (the DNA sequence is shown in SEQ ID NO.41); an E2 (the DNA sequence is shown in SEQ ID NO.42 in this example); a 5′ homology arm sequence (the DNA sequence is shown in SEQ ID NO.54); an IRES sequence (the DNA sequence is shown in SEQ ID NO.38); an IL-2 signal peptide (the DNA sequence is shown in SEQ ID NO.56); a urate oxidase coding fragment (the amino acid sequence is shown in SEQ ID NO.8); a 3′ homology arm sequence (the DNA sequence is shown in SEQ ID NO.55); an E1 (the DNA sequence is shown in SEQ ID NO.46 in this example).

The specific process was as follows:
(1) amplifying a target DNA sequence of PBC by using PBC-F1/PBC-R1 primer pair

Reaction system: 0.5 μL of PBC-F1 (as shown in SEQ ID NO.57); 0.5 μL of PBC-R1 (as shown in SEQ ID NO.58); 0.1 ug of a PBC gene synthesis vector; 10 μL of a 2×Takara primeSTAR; and H₂O made up to 20 μL.

Reaction conditions: 98° C. for 10 min; 98° C. for 30 s; 58° C. for 30 s; 72° C. for 30 s; 72° ° C. for 10 min; 35 cycles; and 4° C., 00.
(2) amplifying the vector sequence by using pCIRC-F2/pCIRC-R2 primer pair

Reaction system: 0.5 μL of pCIRC-F2 (as shown in SEQ ID NO.59); 0.5 UL of pCIRC-R2 (as shown in SEQ ID NO. 60); 0.1 ug of a vector; 10 μL of a 2×Takara primeSTAR; and H₂O made up to 20 μL.

Reaction conditions: 98° C. for 10 min; 98° C. for 30 s; 58° C. for 30 s; 72° C. for 30 s; 72° C. for 10 min; 35 cycles; and 4° C., ∞.
(3) Recovering the target PCR product by DNA gel electrophoresis: a 2% DNA agarose gel was formulated, electrophoresis was conducted at 120 V for 30 min, and a PCR product was recovered by using an Omega gel extraction kit, added with 30 μL of RNase-free water to elute the template, and detected for the concentration.
(4) Construction of a recombinant plasmid for expressing PBC by recombinant method: this example was illustrated by taking the ClonExpress II One Step Cloning Kit of Vazyme as an example. Each component was added sequentially according to the following system: 40 ng of a PBC fragment; 80 ng of a vector fragment; 4 μL of a 5×CE II buffer; 2 μL of Exnase II; H₂O made up to 20 μL. The system was mixed uniformly by gently pipetting up and down with a pipettor, and subjected to transient centrifugation to collect a reaction solution to the bottom of a tube. The reaction solution was reacted at 37° C. for 30 min, then cooled to 4° C. or immediately placed on ice for cooling.
(5) Transformation: the recombinant product was added into a DH5a competent cell, mixed slowly and evenly, then placed on ice for 30 min, then subjected to heat shock at 42° C. for 90 s, and immediately placed on ice for 2 min, and the system was added with 1 ml of an LB culture solution and cultured at 37° C. for 45 min. The bacteria solution was evenly coated in an LB solid medium containing antibiotics by using a spreader, and cultured overnight at 37° C.

### 3. Plasmid Linearization

The recombinant plasmid for expressing PBC was cleaved by using type IIS (e.g., BspQ I, etc.). The selected restriction enzyme site was a site containing an E1 terminal sequence. After enzyme cleavage, a terminal of an in vitro transcription template was the terminal of the E1. Reaction conditions: 10 ug of plasmid; 2 μL of BspQ 1; 2 μL of a 10×reaction buffer; and RNase-free water made up to 20 μL, with a total volume of 20 μL; the system was mixed evenly, and then reacted in water bath at 50° C. for 1 h.

An enzyme-cleaved product of the plasmid was recovered by a DNA gel, wherein a 2% DNA agarose gel was formulated, electrophoresis was conducted at 120 V for 30 min, and the enzyme-cleaved product of the plasmid was recovered by using an Omega gel extraction kit, added with 30 μL of RNase-free water to elute the template, and detected for the concentration.

### 4. In Vitro Transcription Reaction

In vitro transcription was conducted with a T7 high yield RNA synthesis kit to generate a mixture containing the circular RNA (including a linear RNA and a circular RNA) as a main ingredient.

### (1) Reaction Conditions of Each Tube:

20 μL of ATP; 20 UL of CTP; 20 μL of GTP; 20 UL of UTP; 10 ug of a linearized plasmid; 20 μL of a 10×Reaction Buffer; 20 μL of T7 Enzyme Mix; and RNase-free water made up to 200 μL; and the system was mixed uniformly and then reacted in a water bath with the reaction conditions of: 37° C. for 120 min; 50° C. for 20 min; and 4° C., ∞.

If a single enzyme was used for the in vitro transcription reaction, the reaction system was as follows:

20 μL of ATP; 20 μL of CTP; 20 μL of GTP; 20 μL of UTP; 10 ug of a linearized plasmid; 20 μL of a 10×Reaction Buffer; 2.5 KU of a T7 RNA polymerase; 0.4 U of PPase; 200 U of an RNase inhibitor; RNase-free water made up to 200 μL; and the system was mixed uniformly and then reacted in a water bath with the reaction conditions of: 37° C. for 120 min; and 50° C. for 20 min.

### (2) Digestion by DNase I

The system was treated with DNase I for 15 minutes to remove a template DNA; with reaction conditions of each tube of: 10 μL of DNase I (1 U/μL) being added into the IVT product, mixed uniformly and then reacted at 37° C. for 15 min; and the reaction product was stored at −20° C. for a short time, or directly subjected to a purification step of the circular RNA.

### 5. RNA Purification

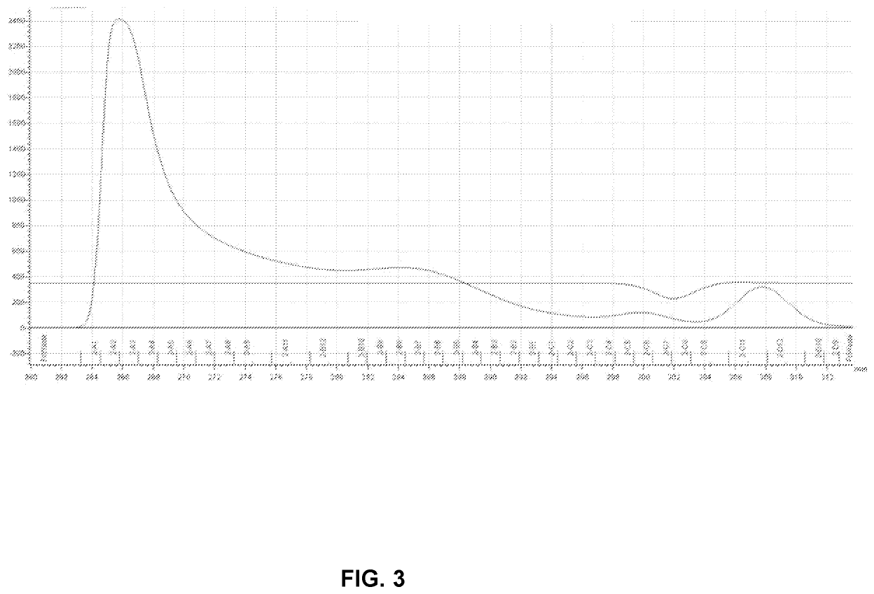

The circular RNA was purified by using an SEC method. SEC conditions: Seplife 6FF, 10 mM Tris-HCl, 150 mM NaCl, 1 mM EDTA, pH 6.0, 60 cm/h, and eluting with RNase free water or a sodium acetate solution. Results were as shown in FIG. 3, and the IVT product mainly included three products: a linear RNA, a circular RNA and a self-splicing product intron RNA.

### 6. Encapsulating Circular RNA by LNP

Taking SM102 as an example, respective components of LNP (SM102: DSPC: Cholesterol PEG2000) were dissolved in ethanol respectively in a ratio of 50:10:38.5:1.5. The circular RNA was dissolved in a sodium acetate solution. Liposomes and RNAs passed through a microfluidic instrument at a rate of 3:1 and were collected. The RNA encapsulated by LNP was diluted by using PBS, and subjected to fluid exchange in a manner such as TFF, or subjected to concentration and fluid exchange by using an ultrafiltration tube. The products encapsulated by LNP could be stored at 4° C. for a short time and at −80° C. for a long time. Detailed information of the encapsulated circular RNAs CVB3-IL2-PBC and IRES9-IL2-PBC was as shown in Table 1.

TABLE 1
information related to circular RNAs

| | Content | Volume | Encapsulation | Particle | |
|---|---|---|---|---|---|
| Name | (mg/ml) | (ml) | rate | size (nm) | PDI |
| CVB3-IL2-PBC | 0.12 | 10 | 92.6% | 125.48 | 0.111 |
| IRES9-IL2-PBC | 0.2 | 7 | 92.1% | 135.51 | 0.156 |

Circular RNA CVB3-IL2-PBC was hereinafter referred to as CVB3; and circular RNA IRES9-IL2-PBC was hereinafter referred to as IRES9.

### Example 2 Evaluation of Therapeutic Effect of Circular RNAs on Hyperuricemia

### 1. Experimental Method

### (1) Experimental Scheme Design

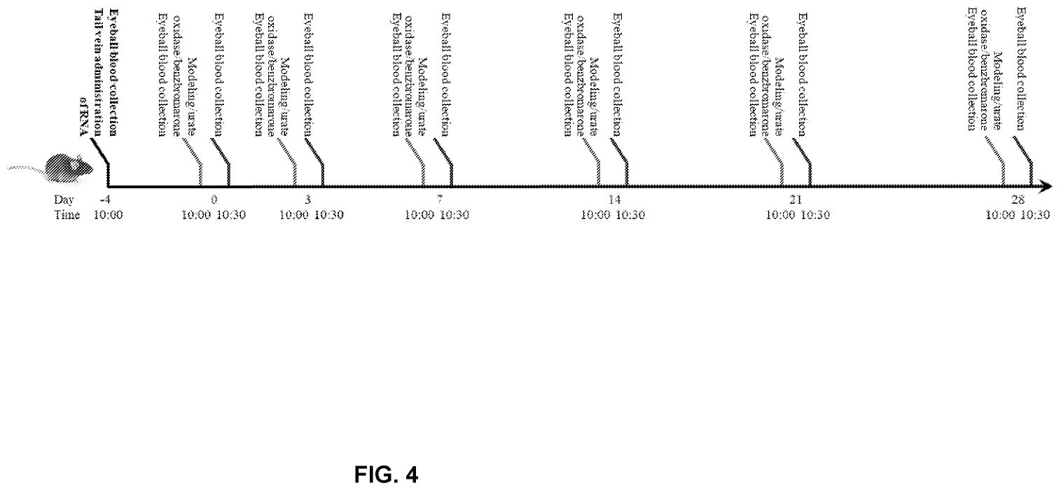

See FIG. 4 for detailed information of administration, model establishment (or modeling) and sampling schemes.
a. a negative control group (6 mice), this model was established by administration of normal saline; b. a hyperuricemia group (6 mice), this model was established by oral gavage of the described above hypoxanthine suspension in combination with intraperitoneal injection of the ethambutol solution; c. a group with a high dose of CVB3 (6 mice, 50 ug/mouse); d. a group with a low dose of CVB3 (6 mice, 10 μg/mouse); e. a group with a high dose of IRES9 (6 mice, 50 μg/mouse); f. a group with a low dose of IRES9 (6 mice, 10 μg/mouse), wherein the groups c, d, e and f were given at a single dose for 4 d and these models were established by oral gavage of the hypoxanthine suspension in combination with intraperitoneal injection of the ethambutol solution; g. a group with benzbromarone (6 mice), this model was established by oral gavage of the hypoxanthine suspension in combination with intraperitoneal injection of the ethambutol solution 4 hours later the oral gavage of the benzbromarone suspension each time; and h. a group with the urate oxidase (6 mice), this model was established by oral gavage of the hypoxanthine suspension in combination with intraperitoneal injection of the ethambutol solution after the tail vein injection of the urate oxidase solution.

Both CVB3 and IRES9 were administered to the mice by single tail vein injection. The benzbromarone was administered by gavage at 30 mg/kg 4 h before model establishment. The urate oxidase was administered at tail vein at 10 mg/kg. The models were established by oral gavage of the hypoxanthine at 1000 mg/kg in combination with intraperitoneal injection of the ethambutol at 250 mg/kg. 30 min after each administration of the hypoxanthine and ethambutol, blood was collected from the orbit and serum was separated for preservation. See Table 2 for specific grouping.

TABLE 2
animal groups, model establishment, and administration

| Groups | Model establishment | Treatment (administration) |
|---|---|---|
| Negative | Normal diet | saline solution |
| control group | | |
| Hyperuricemia | Hypoxanthine: | saline solution |
| group | 1000 mg/kg, | |
| | gavage; ethambutol: 250 | |
| | mg/kg, intraperitoneal | |
| | injection | |
| Group | Hypoxanthine: | 50 μg/mouse, |
| administrated | 1000 mg/kg, | once tail vein |
| with a high | gavage; ethambutol: 250 | injection, 3 days |
| dose of CVB3 | mg/kg, intraperitoneal | before model |
| | injection | establishment |
| Group | Hypoxanthine: | 10 μg/mouse, |
| administrated | 1000 mg/kg, | once tail vein |
| with a low dose | gavage; ethambutol: 250 | injection, 3 days |
| of CVB3 | mg/kg, intraperitoneal | before model |
| | injection | establishment |
| Group | Hypoxanthine: | 50 μg/mouse, |
| administrated | 1000 mg/kg, | once tail vein |
| with a high | gavage; ethambutol: 250 | injection, 3 days |
| dose of IRES9 | mg/kg, intraperitoneal | before model |
| | injection | establishment |
| Group | Hypoxanthine: | 10 μg/mouse, |
| administrated | 1000 mg/kg, | once tail vein |
| with a low dose | gavage; ethambutol: 250 | injection, 3 days |
| of IRES9 | mg/kg, intraperitoneal | before model |
| | injection | establishment |
| Group | Hypoxanthine: | 30 mg/kg of |
| administrated | 1000 mg/kg, | benzbromarone |
| with | gavage; ethambutol: 250 | administrated once by |
| benzbromarone | mg/kg, intraperitoneal | gavage, 4 hours |
| | injection | before model |
| | | establishment |
| Group | Hypoxanthine: | 10 mg/kg of |
| administrated | 1000 mg/kg, | urate oxidase, |
| with urate | gavage; ethambutol: 250 | administrated with tail vein |
| oxidase | mg/kg, intraperitoneal | injection once simultaneously |
| | injection | with model establishment |

### (2) Determination of Content of Uric Acid in Mice

For the experimental steps, please refer to the instruction of CheKine™ Micro Uric Acid (UA) Assay Kit (Cat #: KTB1510 Size: 48T/96T). The experimental sample was serum, which was diluted by Extraction Buffer, for example, 7 μl of the serum sample was mixed with 63 μl of Extraction Buffer (i.e., 10 times dilution). Then 60 μl of the diluted sample was taken and reacted with 150 μl of a working reagent I A at 37° C. for 30 min, and then determined for an absorbance value at a wavelength of 505 nm (OD505 nm). The absorbance value was converted into the content of uric acid by using the equation of UA (μmol/ml)=CStandard×(ATest−ABlank)+(AStandard−ABlank)=5×(ATest−ABlank)+(AStandard−ABlank). The calculated content of uric acid multiplies by 10 (the dilution) to obtain the uric acid in the sample.

### (3) Determination of Urate Oxidase Activity in Mice

For the experimental steps, please refer to the instruction of a urate oxidase activity detection kit (Cat #: BC4435 Size: 100T/48S). The experimental sample was a fresh liver tissue. 0.1 g of a liver tissue was weighed, added with 1 mL of an extracting solution, homogenized in ice bath, and then centrifuged at 10,000 rpm at 4° C. for 10 min. The supernatant was taken and placed on ice. 4 μl of the supernatant sample to be tested+36 μl of the extracting solution, i.e., 10-fold dilution of the sample was created, and then 30 μl of the diluted sample was taken and reacted with 170 μl of a working solution A at 37° C. for 30 min, and used to determine the absorbance value at a wavelength of 505 nm (OD505 nm). The absorbance value was converted into the urate oxidase activity by using the equation of urate oxidase activity (U/g mass)=determination of ΔA÷(AA standard+C standard)×V sample÷(W×V sample÷V extraction)÷T=determination of Δ÷ΔA standard÷W. The calculated value multiplies by 10 (the dilution) to obtain the urate oxidase activity in the sample.

### (4) Detection of Immunogenicity

1) plate coating: a 96-hole high adsorption ELISA plate was taken and coated with a urate oxidase at a concentration of 100 ng/hole at 37° C. for 2 h and at 4° C. overnight; 2) plate washing: each well was washed once by adding 200 μl of a washing solution (PBS containing 0.1% tween-20) and dried; 3) blocking: each well was added with 200 μl of a blocking solution (PBS containing 5% skimmed milk powder) at 4° C. overnight; 4) plate washing: each well was washed for 3 times by adding 200 μl of a washing solution (PBS containing 0.1% tween-20) and dried; 5) sample injecting: the serum sample was diluted and then incubated according to 100 μl/well at 37° C. for 1 h; 6) plate washing: each well was washed for 3 times by adding 200 μl of a plate washing solution (PBS containing 0.1% tween-20) and dried; 7) addition of enzyme-labeled secondary antibody: a corresponding well was added with 100 μl of an HRP-labeled secondary antibody (goat anti-mouse antibody at 1:5000) and incubated at 37° C. for 1 h; 8) plate washing: each well was washed for 5 times by adding 200 μl of the plate washing solution (PBS containing 0.1% tween-20) and dried; 9) addition of substrate: a solution A and a solution B were taken and mixed uniformly, then each well was added with 100 μl of the mixed substrate with protection away from light, and the plate was sealed with a plate sealer and incubated at room temperature for 3-5 minutes; 10) termination: each well was added with 50 μl of a termination solution to terminate the reaction, and put into an microplate reader; and 11) reading: the plate was mixed uniformly by the shaking function of the microplate reader, and read at a working wavelength of 450 nm and a reference wavelength of 620 nm.

### 2. Processing of Test Data

All absorbance data were acquired and processed by Microplate Reader, and calculated and counted by Microsoft Excel 2019 (e.g., mean, standard deviation, etc.). The data was analyzed by GraphPad Prism software, and plotted by taking time as the horizontal coordinate, and taking the content of uric acid or urate oxidase activity as the vertical coordinate. Acceptance criteria of test data: CV≤15% was among any two groups of data of a standard curve, QC samples and samples to be tested; the RD between the concentration results calculated according to two dilutions of each sample was ≤20%, and the result was expressed as a mean.

### 3. Experimental Results

### (1) Experimental Grouping and Animal Number

48 C57 mice were purchased on Aug. 18, 2022, fed adaptively in the laboratory for 4 days, and grouped and numbered on August 22nd. Then the group administrated with the circular RNA expressing the urate oxidase was injected at tail vein once with the specific dosage as shown in Table 2. The first modeling and blood collection was conducted on August 25th, the second modeling and blood collection was conducted on August 29th, the third modeling and blood collection was conducted on September 5th, the fourth modeling and blood collection was conducted on September 12th, the fifth modeling and blood collection was conducted on September 19th, and the sixth modeling and blood collection was conducted on September 26th.

### (2) Detection of Urate Oxidase Activity in Liver Tissue of Mice

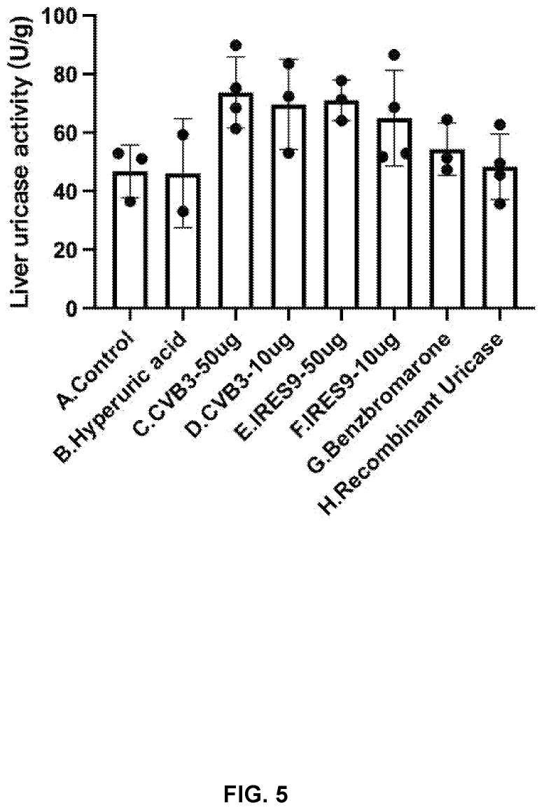

The urate oxidase activity was detected by using a fresh liver tissue taken during animal dissection. After the sixth modeling and sampling was completed, mice were sacrificed by cervical dislocation, and the livers and kidneys of mice in each group were collected. Some of the mice were perfused after anesthesia, and then the livers and kidneys were collected. See Table 3 and FIG. 5 for experimental data. The results showed that compared with the control group, the urate oxidase activity in the liver of mice in the group administrated with a high dose of CVB3 was significantly increased, with P=0.0237; the urate oxidase activity in the liver of mice in the group administrated with a high dose of IRES9 was significantly increased, with P=0.0208; and the urate oxidase activity in the liver of mice in each group administrated with CVB3 and IRES9 was significantly higher than that in the group administrated with the urate oxidase; which showed that the circular RNA for expressing a urate oxidase of the present disclosure can significantly improve the level of urate oxidase.

TABLE 3
Detection of urate oxidase activity in mouse liver

| | | | Relative to the |
|---|---|---|---|
| | Mean ± standard | Relative to the | Hyperuricemia |
| Groups | deviation | control group | group |
| Negative control | 46.79 ± 9.00 | 0.00% | 1.42% |
| group | | | |
| Hyperuricemia | 46.14 ± 18.57 | −1.40% | 0.00% |
| group | | | |
| Group | 73.72 ± 12.11 | 57.54% | 59.79% |
| administrated with a | | | |
| high dose of CVB3 | | | |
| Group | 69.62 ± 15.43 | 48.77% | 50.89% |
| administrated with a | | | |
| low dose of CVB3 | | | |
| Group | 71.04 ± 6.89 | 51.81% | 53.97% |
| administrated with a | | | |
| high dose of IRES9 | | | |
| Group | 64.94 ± 16.33 | 38.77% | 40.75% |
| administrated with a | | | |
| low dose of IRES9 | | | |
| Group | 54.34 ± 8.92 | 16.12% | 17.77% |
| administrated with | | | |
| benzbromarone | | | |
| Group | 48.35 ± 11.22 | 3.32% | 4.79% |
| administrated with | | | |
| urate oxidase | | | |

### (3) Detection of Content of Uric Acid in Serum of Mice

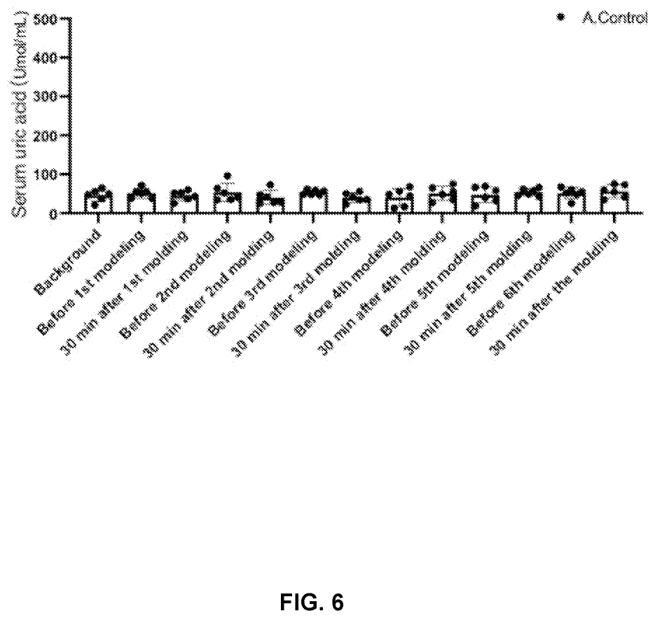

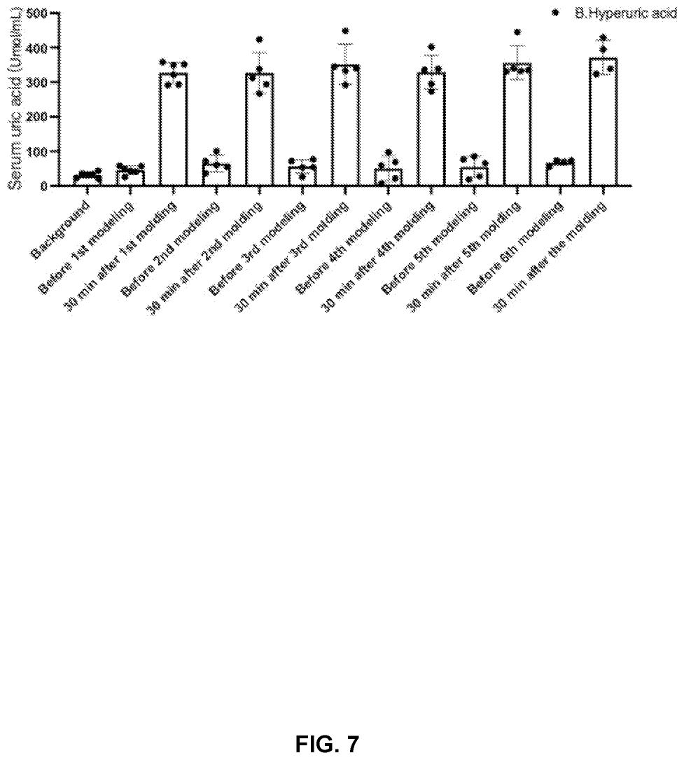

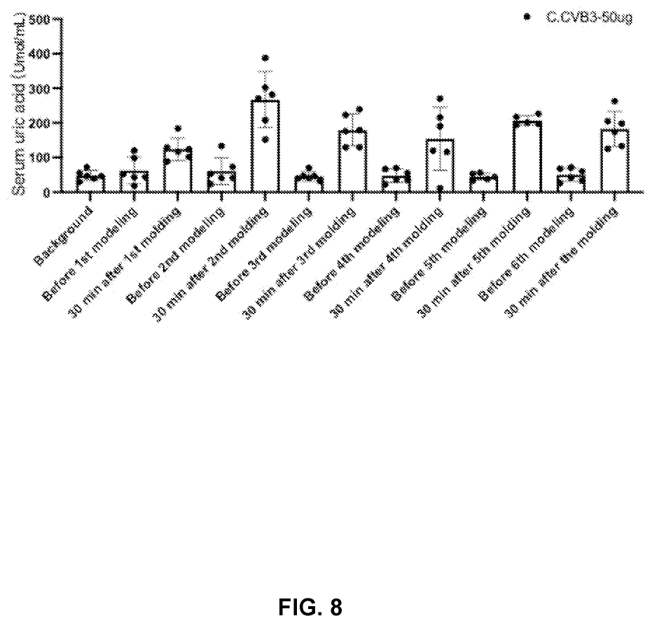

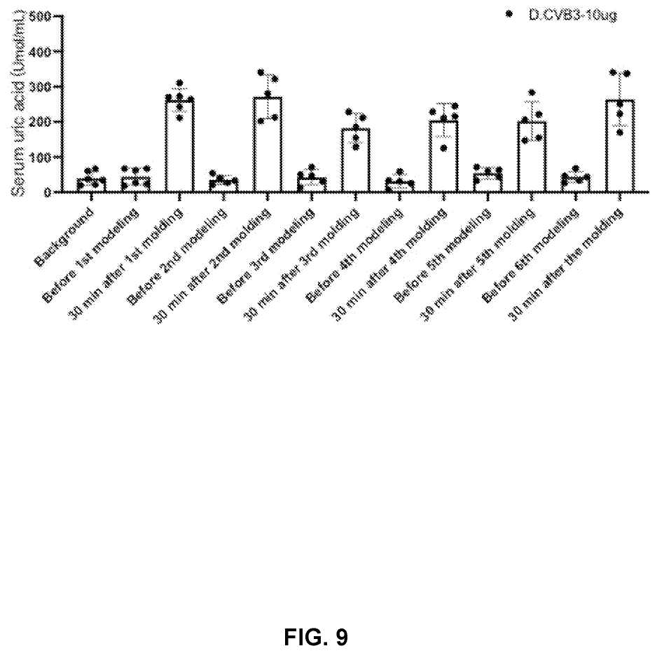

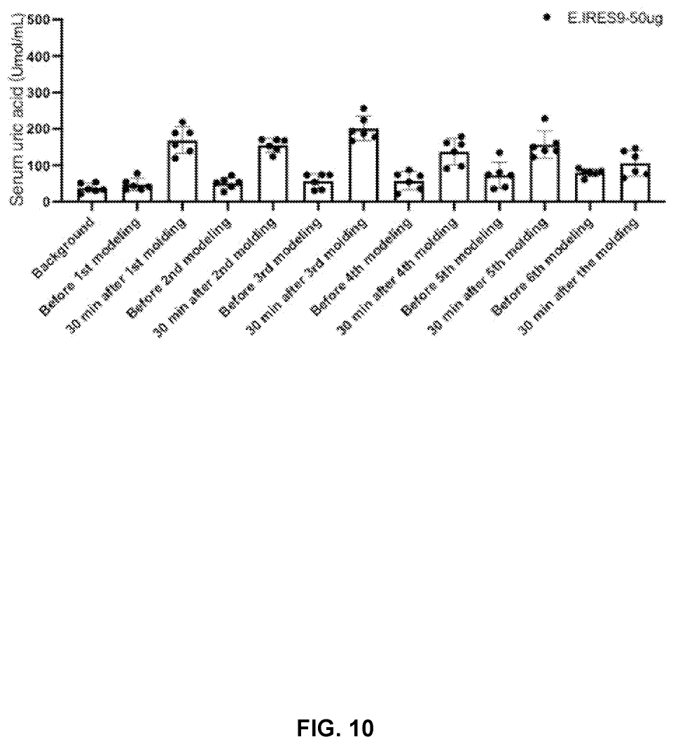

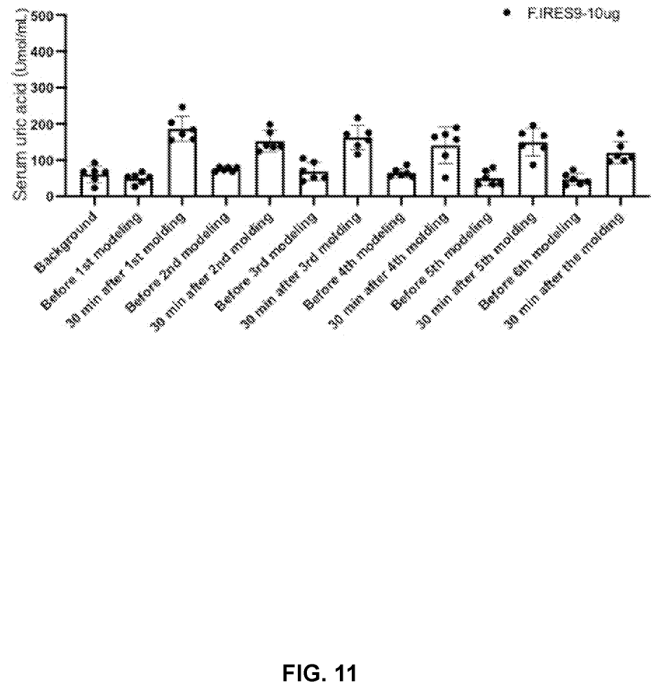

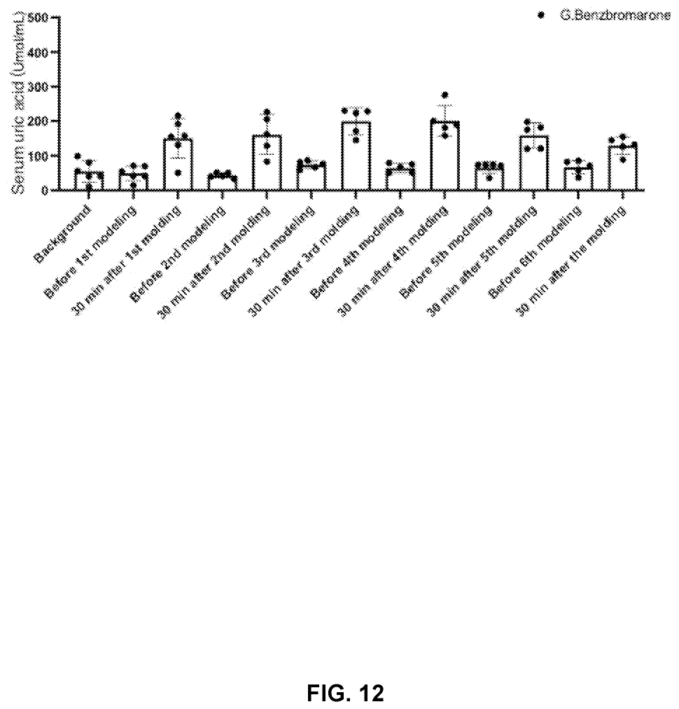

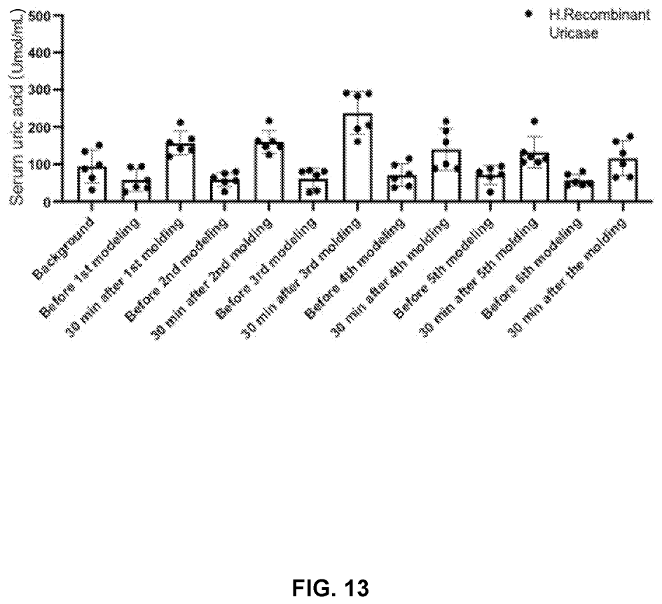

The content of uric acid was detected in the serum sample, the detection value of black was 0.461, and the detection value of the standard was 0.535. See Table 4 and FIGS. 6-13 for the summarized uric acid data. The results showed that the content of uric acid in the serum samples of both groups administrated with a high dose of and a low dose of CVB3 was lower than that in the control group after modeling, and the content of the uric acid was reduced by 20.08% to 62.21% compared with that in the Hyperuricemia group. The content of uric acid in the serum samples of the both groups administrated with a high dose of and a low dose of IRES9 was lower than that in the control group after modeling, and the content of uric acid was reduced by 42.90% to 71.67% in each group compared with that in the Hyperuricemia group. The results demonstrate that the circular RNA described in the present disclosure effectively expresses urate oxidase, leading to prolonged effectiveness and reduced frequency of medication. Notably, a single injection of the circular RNA can maintain low uric acid levels for up to 28 days.

TABLE 4
content of uric acid in serum (Umol/mL)

| | | | | Reduced relative |
|---|---|---|---|---|
| | Basic | | 30 min after D1 | to the Hyperuricemia |
| Groups | value | D1 modeling | modeling | group |
| Negative | 46.17 ± 14.83 | 51.80 ± 12.25 | 45.05 ± 12.38 | |
| control group | | | | |
| Hyperuricemia | 31.53 ± 8.69 | 45.50 ± 12.06 | 327.48 ± 29.89 | |
| group | | | | |
| Group | 48.20 ± 14.58 | 62.61 ± 38.61 | 123.76 ± 32.91 | −62.21% |
| administrated | | | | |
| with a high | | | | |
| dose of CVB3 | | | | |
| Group | 39.98 ± 19.60 | 44.03 ± 23.52 | 261.71 ± 32.93 | −20.08% |
| administrated | | | | |
| with a low dose | | | | |
| of CVB3 | | | | |
| Group | 37.16 ± 12.47 | 46.96 ± 16.94 | 168.24 ± 36.92 | −48.63% |
| administrated | | | | |
| with a high | | | | |
| dose of IRES9 | | | | |
| Group | 61.26 ± 23.24 | 49.21 ± 14.37 | 186.26 ± 34.59 | −43.12% |
| administrated | | | | |
| with a low dose | | | | |
| of IRES9 | | | | |
| Group | 54.62 ± 31.45 | 48.87 ± 21.22 | 150.23 ± 57.44 | −54.13% |
| administrated | | | | |
| with | | | | |
| benzbromarone | | | | |
| Group | 94.26 ± 44.34 | 57.55 ± 29.40 | 156.53 ± 31.81 | −52.20% |
| administrated | | | | |
| with urate | | | | |
| oxidase | | | | |
| | | | | Reduced relative |
| | Basic | | 30 min after D3 | to the Hyperuricemia |
| Groups | value | D3 modeling | modeling | group |
| Negative | 46.17 ± 14.83 | 53.60 ± 23.58 | 40.65 ± 18.07 | |
| control group | | | | |
| Hyperuricemia | 31.53 ± 8.69 | 64.59 ± 23.96 | 326.89 ± 59.97 | |
| group | | | | |
| Group | 48.20 ± 14.58 | 60.81 ± 39.00 | 266.89 ± 80.72 | −18.35% |
| administrated | | | | |
| with a high | | | | |
| dose of CVB3 | | | | |
| Group | 39.98 ± 19.60 | 35.14 ± 12.83 | 271.49 ± 62.50 | −16.95% |
| administrated | | | | |
| with a low dose | | | | |
| of CVB3 | | | | |
| Group | 37.16 ± 12.47 | 50.68 ± 15.50 | 154.73 ± 18.44 | −52.67% |
| administrated | | | | |
| with a high | | | | |
| dose of IRES9 | | | | |
| Group | 61.26 ± 23.24 | 75.11 ± 6.23 | 152.14 ± 28.85 | −53.46% |
| administrated | | | | |
| with a low dose | | | | |
| of IRES9 | | | | |
| Group | 54.62 ± 31.45 | 43.24 ± 7.54 | 161.22 ± 57.73 | −50.68% |
| administrated | | | | |
| with | | | | |
| benzbromarone | | | | |
| Group | 94.26 ± 44.34 | 59.01 ± 19.11 | 160.36 ± 30.76 | −50.94% |
| administrated | | | | |
| with urate | | | | |
| oxidase | | | | |
| | | | | Reduced relative |
| | Basic | | 30 min after D7 | to the Hyperuricemia |
| Groups | value | D7 modeling | modeling | group |
| Negative | 46.17 ± 14.83 | 53.83 ± 6.32 | 39.98 ± 11.95 | |
| control group | | | | |
| Hyperuricemia | 31.53 ± 8.69 | 56.08 ± 20.15 | 351.62 ± 58.12 | |
| group | | | | |
| Group | 48.20 ± 14.58 | 45.95 ± 13.12 | 179.32 ± 45.74 | −49.00% |
| administrated | | | | |
| with a high | | | | |
| dose of CVB3 | | | | |
| Group | 39.98 ± 19.60 | 42.43 ± 21.72 | 181.68 ± 40.85 | −48.33% |
| administrated | | | | |
| with a low dose | | | | |
| of CVB3 | | | | |
| Group | 37.16 ± 12.47 | 56.08 ± 20.63 | 200.79 ± 33.33 | 42.90% |
| administrated | | | | |
| with a high | | | | |
| dose of IRES9 | | | | |
| Group | 61.26 ± 23.24 | 69.03 ± 25.72 | 162.50 ± 34.06 | −53.79% |
| administrated | | | | |
| with a low dose | | | | |
| of IRES9 | | | | |
| Group | 54.62 ± 31.45 | 73.92 ± 11.11 | 199.86 ± 39.02 | −43.16% |
| administrated | | | | |
| with | | | | |
| benzbromarone | | | | |
| Group | 94.26 ± 44.34 | 61.57 ± 27.40 | 237.50 ± 57.15 | −32.46% |
| administered | | | | |
| with urate | | | | |
| oxidase | | | | |
| | | | | Reduced relative |
| | Basic | | 30 min after D14 | to Hyperuricemia |
| Groups | value | D14 modeling | modeling | group |
| Negative | 46.17 ± 14.83 | 41.10 ± 21.96 | 50.90 ± 17.96 | |
| control group | | | | |
| Hyperuricemia | 31.53 ± 8.69 | 50.68 ± 36.56 | 328.92 ± 49.29 | |
| group | | | | |
| Group | 48.20 ± 14.58 | 47.64 ± 19.16 | 153.94 ± 91.09 | −53.20% |
| administrated | | | | |
| with a high | | | | |
| dose of CVB3 | | | | |
| Group | 39.98 ± 19.60 | 31.08 ± 18.49 | 204.87 ± 46.53 | −37.71% |
| administrated | | | | |
| with a low dose | | | | |
| of CVB3 | | | | |
| Group | 37.16 ± 12.47 | 56.98 ± 25.17 | 137.05 ± 35.96 | −58.33% |
| administrated | | | | |
| with a high | | | | |
| dose of IRES9 | | | | |
| Group | 61.26 ± 23.24 | 64.86 ± 12.55 | 140.77 ± 50.60 | −57.20% |
| administrated | | | | |
| with a low dose | | | | |
| of IRES9 | | | | |
| Group | 54.62 ± 31.45 | 64.46 ± 13.54 | 200.95 ± 44.59 | −38.91% |
| administered | | | | |
| with | | | | |
| benzbromarone | | | | |
| Group | 94.26 ± 44.34 | 71.17 ± 30.78 | 140.32 ± 55.41 | −57.34% |
| administrated | | | | |
| with urate | | | | |
| oxidase | | | | |
| | | | | Reduced relative |
| | Basic | | 30 min after D21 | to the Hyperuricemia |
| Groups | value | D21 modeling | modeling | group |
| Negative | 46.17 ± 14.83 | 47.64 ± 20.44 | 53.94 ± 9.38 | |
| control group | | | | |
| Hyperuricemia | 31.53 ± 8.69 | 54.87 ± 30.17 | 356.53 ± 49.70 | |
| group | | | | |
| Group | 48.20 ± 14.58 | 44.86 ± 9.83 | 207.03 ± 13.94 | −41.93% |
| administrated | | | | |
| with a high | | | | |
| dose of CVB3 | | | | |
| Group | 39.98 ± 19.60 | 54.19 ± 15.86 | 202.30 ± 55.17 | −43.26% |
| administrated | | | | |
| with a low dose | | | | |
| of CVB3 | | | | |
| Group | 37.16 ± 12.47 | 72.52 ± 35.65 | 156.64 ± 37.37 | −56.07% |
| administrated | | | | |
| with a high | | | | |
| dose of IRES9 | | | | |
| Group | 61.26 ± 23.24 | 50.23 ± 20.72 | 149.32 ± 38.16 | −58.12% |
| administrated | | | | |
| with a low dose | | | | |
| of IRES9 | | | | |
| Group | 54.62 ± 31.45 | 65.14 ± 16.80 | 159.05 ± 36.38 | −55.39% |
| administrated | | | | |
| with | | | | |
| benzbromarone | | | | |
| Group | 94.26 ± 44.34 | 71.28 ± 24.92 | 132.21 ± 41.94 | −62.92% |
| administrated | | | | |
| with urate | | | | |
| oxidase | | | | |
| | | Model | 30 min after | Reduced relative |
| | Basic | establishment | model establishment | to the Hyperuricemia |
| Groups | value | at D28 | at D28 | model group |
| Negative | 46.17 ± 14.83 | 51.46 ± 14.35 | 56.53 ± 16.64 | |
| control group | | | | |
| Hyperuricemia | 31.53 ± 8.69 | 67.57 ± 8.33 | 371.62 ± 49.34 | |
| model group | | | | |
| Group | 48.20 ± 14.58 | 50.11 ± 18.92 | 182.86 ± 51.00 | −50.79% |
| administrated | | | | |
| with a high | | | | |
| dose of CVB3 | | | | |
| Group | 39.98 ± 19.60 | 43.51 ± 15.63 | 263.92 ± 74.26 | −28.98% |
| administrated | | | | |
| with a low dose | | | | |
| of CVB3 | | | | |
| Group | 37.16 ± 12.47 | 79.39 ± 10.30 | 105.29 ± 35.24 | −71.67% |
| administrated | | | | |
| with a high | | | | |
| dose of IRES9 | | | | |
| Group | 61.26 ± 23.24 | 47.41 ± 16.09 | 120.61 ± 29.72 | −67.54% |
| administrated | | | | |
| with a low dose | | | | |
| of IRES9 | | | | |
| Group | 54.62 ± 31.45 | 66.76 ± 19.79 | 129.19 ± 25.29 | −65.24% |
| administrated | | | | |
| with | | | | |
| benzbromarone | | | | |
| Group | 94.26 ± 44.34 | 56.87 ± 15.97 | 116.22 ± 46.89 | −68.73% |
| administrated | | | | |
| with urate | | | | |
| oxidase | | | | |

### (4) Detection of Anti-Urate Oxidase Antibody in Serum of Mice

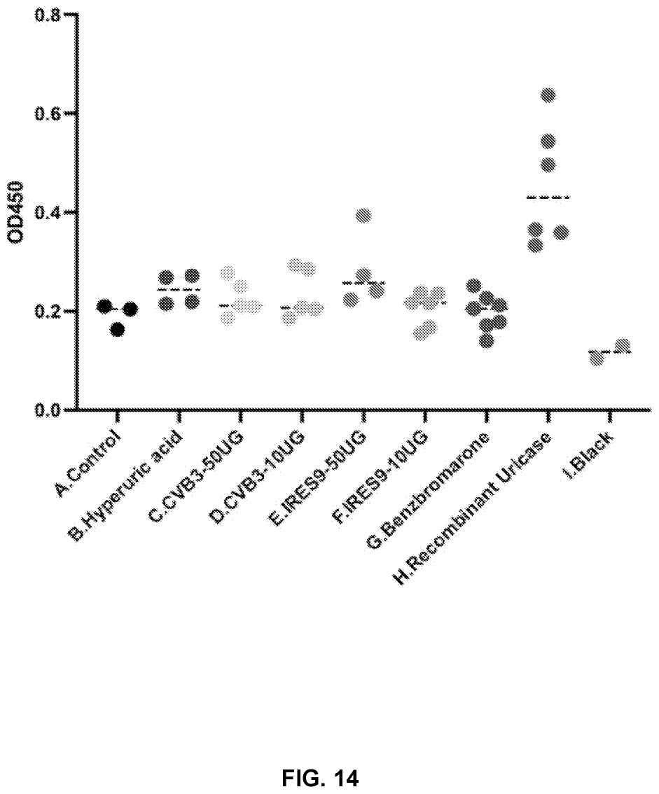

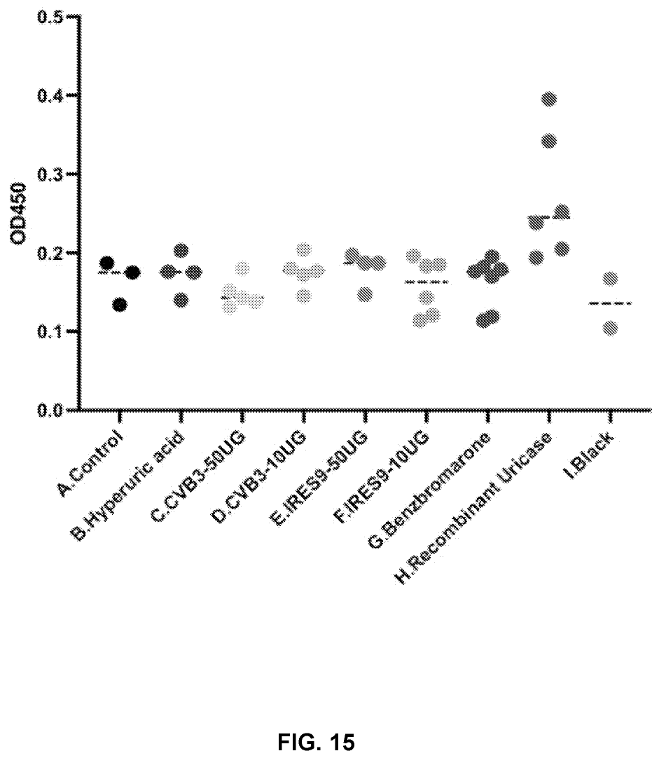

The antibody titer against urate oxidase in the serum sample of each group was detected by indirect ELISA. FIG. 14 shows the antibody titer for 10-fold dilution of the serum samples, and FIG. 15 shows the antibody titer for 100-fold dilution. At 28 days after the corresponding intervention treatment in each mouse group, the serum was diluted 10 times and 100 times respectively. An elevated level of reactive antibody against urate oxidase was detected in the serum sample of the group administered with urate oxidase compared to the blank and control groups, while no significant anti-urate oxidase antibody was detected in the other groups. This indicates that the circular RNA in the present disclosure demonstrated good safety.

### (5) Conclusion

1) In this example, the mouse model with acute hyperuricemia was established by gavage of 1,000 mg/kg of hypoxanthine in combination with intraperitoneal injection of 250 mg/kg of ethambutol, so as to meet the experimental requirements. 2) Urate oxidase activities in fresh liver tissue samples were detected by a colorimetric method, wherein the urate oxidase activities in the liver tissue samples of the group administrated with a high dose of CVB3 was higher than that in the control group, and has a statistical difference with p=0.0237; the urate oxidase activities in the liver samples of the group administrated with a high dose of IRES9 was higher than that in the control group and has a statistical difference with p=0.0208; no significant difference was found in comparison among the other groups, but in clinical symptoms, compared with the control group, a uric acid in each group was all reduced. 3) The content of uric acid in the serum sample was detected by a kit, the detection value of the black was 0.461, and the detection value of the standard was 0.535, wherein the content of uric acid in serum sample in each group administrated with CVB3 after model establishment was lower than that in the control group, and has a statistical difference; and the content of uric acid in serum sample in each group administrated with IRES9 after model establishment was lower than that in the control group, and has a statistical difference. 4) A reactive antibody against the urate oxidase was detected in the serum sample of the group administrated with the urate oxidase, but no obvious anti-urate oxidase antibody was detected in other groups, indicating that the drug (the circular RNA) had long-term safety and effectiveness after administration.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. Furthermore, the particular features, structures or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

SEQUENCE LISTING

SEQ ID NO: 1 urate oxidase coding fragment from Sus scrofa
TYKKNDEVEFVRTGYGKDMIKVLHIQRDGKYHSIKEVATSVQLTLSSKKDYLHGDN
SDVIPTDTIKNTVNVLAKFKGIKSIETFAVTICEHFLSSFKHVIRAQVYVEEVPWKRFE
KNGVKHVHAFIYTPTGTHFCEVEQIRNGPPVIHSGIKDLKVLKTTQSGFEGFIKDQFT
TLPEVKDRCFATQVYCKWRYHQGRDVDFEATWDTVRSIVLQKFAGPYDKGEYSPS
VQKTLYDIQVLSLSRVPEIEDMEISLPNIHYFNIDMSKMGLINKEEVLLPLDNPYGKIT
GTVKRKLS
SEQ ID NO: 2 urate oxidase coding fragment from Sus scrofa including signal
positioning peptide
TYKKNDEVEFVRTGYGKDMIKVLHIQRDGKYHSIKEVATSVQLTLSSKKDYLHGDN
SDVIPTDTIKNTVNVLAKFKGIKSIETFAVTICEHFLSSFKHVIRAQVYVEEVPWKRFE
KNGVKHVHAFIYTPTGTHFCEVEQIRNGPPVIHSGIKDLKVLKTTQSGFEGFIKDQFT
TLPEVKDRCFATQVYCKWRYHQGRDVDFEATWDTVRSIVLQKFAGPYDKGEYSPS
VQKTLYDIQVLSLSRVPEIEDMEISLPNIHYFNIDMSKMGLINKEEVLLPLDNPYGKIT
GTVKRKLSSRL
SEQ ID NO. 3 urate oxidase coding fragment from Papio anubis
MADYHNNYKKNDELEFVRTGYGKDMVKVLHIQRDGKYHSIKEVATSVQLTLSSKKD
YLHGDNSDIIPTDTIKNTVHVLAKFKGIKSIEAFGVNICEYFLSSFNHVIRAQVYVEEIP
WKRLEKNGVKHVHAFIHTPTGTHFCEVEQLRSGPPVIHSGIKDLKVLKTTQSGFEG
FIKDQFTTLPEVKDRCFATQVYCKWRYHQCRDVDFEATWGTIRDLVLEKFAGPYDK
GEYSPSVQKTLYDIQVLSLSRVPEIEDMEISLPNIHYFNIDMSKMGLINKEEVLLPLDN
PYGKITGTVKRKLS
SEQ ID NO. 4 urate oxidase coding fragment from Porcine
MAHYRNDYKKNDEVEFVRTGYGKDMIKVLHIQRDGKYHSIKEVATSVQLTLSSKKD
YLHGDNSDVIPTDTIKNTVNVLAKFKGIKSIETFAVTICEHFLSSFKHVIRAQVYVEEV
PWKRFEKNGVKHVHAFIYTPTGTHFCEVEQIRNGPPVIHSGIKDLKVLKTTQSGFEG
FIKDQFTTLPEVKDRCFATQVYCKWRYHQGRDVDFEATWDTVRSIVLQKFAGPYD
KGEYSPSVQKTLYDIQVLTLGQVPEIEDMEISLPNIHYLNIDMSKMGLINKEEVLLPLD
NPYGRITGTVKRKLTSRL
SEQ ID NO. 5 urate oxidase coding fragment from Canis lupus familiaris
MAHYHNDYKKNDEVEFVRTGYGKDMVKVLHIQRDGKYHSIKEVATSVQLTLSSKK
DYVYGDNSDIIPTDTIKNTVHVLAKFKGIKSIETFAMNICEHFLSSFNHVIRAQVYVEE
VPWKRFEKNGVKHVHAFIHNPTGTHFCEVEQMRSGPPVIHSGIKDLKVLKTTQSGF
EGFIKDQFTTLPEVKDRCFATKVYCKWRYHQGRDVDFEATWDTVRDIVLEKFAGP
YDKGEYSPSVQKTLYDIQVHSLSRVPEMEDMEISLPNIHYFNIDMSKMGLINKEEVL
LPLDNPYGRITGTAKRKLASKL
SEQ ID NO. 6 urate oxidase coding fragment from Bos taurus
MAHYHNDYQKNDEVEFVRTGYGKDMVKVLHIQRDGKYHSIKEVATSVQLTLNSRR
EYLHGDNSDIIPTDTIKNTVQVLAKFKGIKSIETFAMNICEHFLSSFNHVIRVQVYVEE
VPWKRFEKNGVKHVHAFIHTPTGTHFCEVEQLRSGPPVIHSGIKDLKVLKTTQSGF
EGFLKDQFTTLPEVKDRCFATQVYCKWRYHQGRDVDFEATWEAVRGIVLKKFAGP
YDKGEYSPSVQKTLYDIQVLSLSQLPEIEDMEISLPNIHYFNIDMSKMGLINKEEVLLP
LDNPYGRITGTVKRKLTSRL
SEQ ID NO. 7 urate oxidase coding fragment PBC
AHYRNDYKKNDEVEFVRTGYGKDMIKVLHIQRDGKYHSIKEVATSVQLTLSSKKDY
LHGDNSDVIPTDTIKNTVNVLAKFKGIKSIETFAVTICEHFLSSFKHVIRAQVYVEEVP
WKRFEKNGVKHVHAFIYTPTGTHFCEVEQIRNGPPVIHSGIKDLKVLKTTQSGFEGF
IKDQFTTLPEVKDRCFATQVYCKWRYHQGRDVDFEATWDTVRSIVLQKFAGPYDK
GEYSPSVQKTLYDIQVLSLSRVPEIEDMEISLPNIHYFNIDMSKMGLINKEEVLLPLDN
PYGKITGTVKRKLS
SEQ ID NO. 8 urate oxidase coding fragment PBC
MAHYRNDYKKNDEVEFVRTGYGKDMIKVLHIQRDGKYHSIKEVATSVQLTLSSKKD
YLHGDNSDVIPTDTIKNTVNVLAKFKGIKSIETFAVTICEHFLSSFKHVIRAQVYVEEV
PWKRFEKNGVKHVHAFIYTPTGTHFCEVEQIRNGPPVIHSGIKDLKVLKTTQSGFEG
FIKDQFTTLPEVKDRCFATQVYCKWRYHQGRDVDFEATWDTVRSIVLQKFAGPYD
KGEYSPSVQKTLYDIQVLSLSRVPEIEDMEISLPNIHYFNIDMSKMGLINKEEVLLPLD
NPYGKITGTVKRKLS
SEQ ID NO. 9 cDNA sequence of the urate oxidase coding fragment
GCCCACTACAGGAATGATTACAAGAAGAACGATGAGGTGGAGTTCGTGAGAAC
CGGCTACGGCAAGGACATGATCAAGGTGCTGCACATCCAGAGAGACGGCAAGT
ACCACAGCATCAAGGAGGTGGCCACCTCCGTGCAGCTGACACTGAGCTCCAAG
AAGGATTACCTGCACGGCGATAATAGCGATGTGATCCCCACAGACACCATCAA
GAACACAGTGAACGTGCTGGCCAAGTTTAAGGGCATCAAGTCCATCGAGACAT
TTGCCGTGACCATCTGTGAGCACTTCCTGTCCAGCTTTAAGCACGTGATCAGAG
CCCAGGTGTACGTGGAGGAGGTGCCTTGGAAGAGGTTCGAGAAGAATGGCGT
GAAGCACGTGCACGCCTTTATCTACACACCCACCGGCACCCACTTCTGCGAGG
TGGAGCAGATCAGAAATGGCCCTCCCGTGATCCACTCCGGCATCAAGGACCTG
AAGGTGCTGAAGACCACCCAGAGCGGCTTCGAGGGCTTCATCAAGGACCAGTT
CACCACCCTGCCCGAGGTGAAGGATAGATGCTTCGCCACACAGGTGTACTGTA
AGTGGAGATACCACCAGGGCAGAGACGTGGACTTCGAGGCCACATGGGACAC
AGTGAGAAGCATCGTGCTGCAGAAGTTTGCCGGCCCTTACGACAAGGGCGAGT
ACAGCCCCTCTGTGCAGAAGACCCTCTATGATATCCAGGTGCTCTCCCTGAGC
CGAGTTCCTGAGATAGAAGATATGGAAATCAGCCTGCCAAACATTCACTACTTC
AATATAGACATGTCCAAAATGGGTCTGATCAACAAGGAAGAGGTCTTGCTGCCA
TTAGACAATCCATATGGAAAAATTACTGGTACAGTCAAGAGGAAGTTGTCTTGA
SEQ ID NO. 10 cDNA sequence of the urate oxidase coding fragment
ATGGCCCACTACAGGAATGATTACAAGAAGAACGATGAGGTGGAGTTCGTGAG
AACCGGCTACGGCAAGGACATGATCAAGGTGCTGCACATCCAGAGAGACGGCA
AGTACCACAGCATCAAGGAGGTGGCCACCTCCGTGCAGCTGACACTGAGCTCC
AAGAAGGATTACCTGCACGGCGATAATAGCGATGTGATCCCCACAGACACCAT
CAAGAACACAGTGAACGTGCTGGCCAAGTTTAAGGGCATCAAGTCCATCGAGA
CATTTGCCGTGACCATCTGTGAGCACTTCCTGTCCAGCTTTAAGCACGTGATCA
GAGCCCAGGTGTACGTGGAGGAGGTGCCTTGGAAGAGGTTCGAGAAGAATGG
CGTGAAGCACGTGCACGCCTTTATCTACACACCCACCGGCACCCACTTCTGCG
AGGTGGAGCAGATCAGAAATGGCCCTCCCGTGATCCACTCCGGCATCAAGGAC
CTGAAGGTGCTGAAGACCACCCAGAGCGGCTTCGAGGGCTTCATCAAGGACCA
GTTCACCACCCTGCCCGAGGTGAAGGATAGATGCTTCGCCACACAGGTGTACT
GTAAGTGGAGATACCACCAGGGCAGAGACGTGGACTTCGAGGCCACATGGGA
CACAGTGAGAAGCATCGTGCTGCAGAAGTTTGCCGGCCCTTACGACAAGGGCG
AGTACAGCCCCTCTGTGCAGAAGACCCTCTATGATATCCAGGTGCTCTCCCTGA
GCCGAGTTCCTGAGATAGAAGATATGGAAATCAGCCTGCCAAACATTCACTACT
TCAATATAGACATGTCCAAAATGGGTCTGATCAACAAGGAAGAGGTCTTGCTGC
CATTAGACAATCCATATGGAAAAATTACTGGTACAGTCAAGAGGAAGTTGTCT
SEQ ID NO. 11 IL2 signal peptide
MYRMQLLSCIALSLALVTNS
SEQ ID NO. 12 HLA signal peptide
MAVMAPRTLVLLLSGALALTQTWA
SEQ ID NO. 13 LRG1 signal peptide
MSSWSRQRPKSPGGIQPHVSRTLFLLLLLAASAWG
SEQ ID NO. 14 CHRNA1 signal peptide
MEPWPLLLLFSLCSAGLVLG
SEQ ID NO. 15 APOB signal peptide
MDPPRPALLALLALPALLLLLLAGARA
SEQ ID NO. 16 CST5 signal peptide
MMWPMHTPLLLLTALMVAVA
SEQ ID NO. 17 GALC signal peptide
MTAAAGSAGRAAVPLLLCALLAPGGA
SEQ ID NO. 18 GSN signal peptide
MAPHRPAPALLCALSLALCALSLPVRA
SEQ ID NO. 19 GP1BA signal peptide
MPLLLLLLLLPSPLHP
SEQ ID NO. 20 GZMB signal peptide
MQPILLLLAFLLLPRADA
SEQ ID NO. 21 SERPING1 signal peptide
MASRLTLLTLLLLLLAGDRASS
SEQ ID NO. 22 IL12A signal peptide
MCPARSLLLVATLVLLDHLSLA
SEQ ID NO. 23 IL10 signal peptide
MHSSALLCCLVLLTGVRA
SEQ ID NO. 24 IL1RL1 signal peptide
MGFWILAILTILMYSTAA
SEQ ID NO. 25 INSR signal peptide
MGTGGRRGAAAAPLLVAVAALLLGAAG
SEQ ID NO. 26 KIR2DL1 signal peptide
MSLLVVSMACVGFFLLQGAWP
SEQ ID NO. 27 KLK14 signal peptide
MFLLLTALQVLAIAMTQS
SEQ ID NO. 28 LACRT signal peptide
MKFTTLLFLAAVAGALVYA
SEQ ID NO. 29 LAG3 signal peptide
MWEAQFLGLLFLQPLWVAPVKPLQPGAE
SEQ ID NO. 30 pIRES1
CAGCACGAGCTGCTC
SEQ ID NO. 31 PIRES2
CTTCTTCTTCCTCTT
SEQ ID NO. 32 pIRES3
TCATGCCGGTGCTGG
SEQ ID NO. 33 pIRES4
ACCAAATGGAGAACA
SEQ ID NO. 34 PIRES5
TCAGATGTGCAGACA
SEQ ID NO. 35 PIRES6
CCAGAGCCTGG
SEQ ID NO. 36 PIRES7
CATTGCTAACAATGTCCACT
SEQ ID NO. 37 PIRES8
GCTGAAGCTG
SEQ ID NO. 38 pIRES9
ACTGGAGTTCTGTGACAGGA
SEQ ID NO. 39 pIRES10
TCTTCCTCTT
SEQ ID NO. 40 CVB3
TTAAAACAGCCTGTGGGTTGATCCCACCCACAGGCCCATTGGGCGCTAGCACT
CTGGTATCACGGTACCTTTGTGCGCCTGTTTTATACCCCCTCCCCCAACTGTAA
CTTAGAAGTAACACACACCGATCAACAGTCAGCGTGGCACACCAGCCACGTTTT
GATCAAGCACTTCTGTTACCCCGGACTGAGTATCAATAGACTGCTCACGCGGTT
GAAGGAGAAAGCGTTCGTTATCCGGCCAACTACTTCGAAAAACCTAGTAACACC
GTGGAAGTTGCAGAGTGTTTCGCTCAGCACTACCCCAGTGTAGATCAGGTCGA
TGAGTCACCGCATTCCCCACGGGCGACCGTGGCGGTGGCTGCGTTGGCGGCC
TGCCCATGGGGAAACCCATGGGACGCTCTAATACAGACATGGTGCGAAGAGTC
TATTGAGCTAGTTGGTAGTCCTCCGGCCCCTGAATGCGGCTAATCCTAACTGCG
GAGCACACACCCTCAAGCCAGAGGGCAGTGTGTCGTAACGGGCAACTCTGCA
GCGGAACCGACTACTTTGGGTGTCCGTGTTTCATTTTATTCCTATACTGGCTGC
TTATGGTGACAATTGAGAGATCGTTACCATATAGCTATTGGATTGGCCATCCGG
TGACTAATAGAGCTATTATATATCCCTTTGTTGGGTTTATACCACTTAGCTTGAA
AGAGGTTAAAACATTACAATTCATTGTTAAGTTGAATACAGCAAA
SEQ ID NO. 41 intron
AAATAATTGAGCCTTAAAGAAGAAATTCTTTAAGTGGATGCTCTCAAACTCAGGG
AAACCTAAATCTAGTTATAGACAAGGCAATCCTGAGCCAAGCCGAAGTAGTAAT
TAGTAAGTTAACAATAGATGACTTACAACTAATCGGAAGGTGCAGAGACTCGAC
GGGAGCTACCCTAACGTCAAGACGAGGGTAAAGAGAGAGTCCAATTCTCAAAG
CCAATAGGCAGTAGCGAAAGCTGCAAGAGAATG
SEQ ID NO. 42 E2
AAAATCCGTTGACCTTAAACGGTCGTGTGGGTTCAAGTCCCTCCACCCCCA
SEQ ID NO. 43 E2
AAAATCCGTTGACCTTAAACGGTCGTGTGGGTTCAAGTCC
SEQ ID NO. 44 E2
AAAATCCGTTGACCTTAAACGGTCGTGTGG
SEQ ID NO. 45 E2
AAAATCCGTTGACCTTAAAC
SEQ ID NO. 46 E1
AGACGCTACGGACTT
SEQ ID NO. 47 E1
CTACGGACTT
SEQ ID NO. 48 E1
AGACGCTACGGAGTT
SEQ ID NO. 49 E1
AGACGCTACGGATTT
SEQ ID NO. 50 CVB3-IL2-PBC
AAAATCCGTTGACCTTAAACGGTCGTGTGGGTTCAAGTCCCTCCACCCCCACG
CCGGAAACGCAATAGCCGTTAAAACAGCCTGTGGGTTGATCCCACCCACAGGC
CCATTGGGCGCTAGCACTCTGGTATCACGGTACCTTTGTGCGCCTGTTTTATAC
CCCCTCCCCCAACTGTAACTTAGAAGTAACACACACCGATCAACAGTCAGCGTG
GCACACCAGCCACGTTTTGATCAAGCACTTCTGTTACCCCGGACTGAGTATCAA
TAGACTGCTCACGCGGTTGAAGGAGAAAGCGTTCGTTATCCGGCCAACTACTT
CGAAAAACCTAGTAACACCGTGGAAGTTGCAGAGTGTTTCGCTCAGCACTACCC
CAGTGTAGATCAGGTCGATGAGTCACCGCATTCCCCACGGGCGACCGTGGCG
GTGGCTGCGTTGGCGGCCTGCCCATGGGGAAACCCATGGGACGCTCTAATAC
AGACATGGTGCGAAGAGTCTATTGAGCTAGTTGGTAGTCCTCCGGCCCCTGAA
TGCGGCTAATCCTAACTGCGGAGCACACACCCTCAAGCCAGAGGGCAGTGTGT
CGTAACGGGCAACTCTGCAGCGGAACCGACTACTTTGGGTGTCCGTGTTTCAT
TTTATTCCTATACTGGCTGCTTATGGTGACAATTGAGAGATCGTTACCATATAGC
TATTGGATTGGCCATCCGGTGACTAATAGAGCTATTATATATCCCTTTGTTGGGT
TTATACCACTTAGCTTGAAAGAGGTTAAAACATTACAATTCATTGTTAAGTTGAAT
ACAGCAAAGCCACCATGTACAGGATGCAACTCCTGTCTTGCATTGCACTAAGTC
TTGCACTTGTCACGAATTCGGCCCACTACAGGAATGATTACAAGAAGAACGATG
AGGTGGAGTTCGTGAGAACCGGCTACGGCAAGGACATGATCAAGGTGCTGCAC
ATCCAGAGAGACGGCAAGTACCACAGCATCAAGGAGGTGGCCACCTCCGTGCA
GCTGACACTGAGCTCCAAGAAGGATTACCTGCACGGCGATAATAGCGATGTGA
TCCCCACAGACACCATCAAGAACACAGTGAACGTGCTGGCCAAGTTTAACGGC
ATCAAGTCCATCGAGACATTTGCCGTGACCATCTGTGAGCACTTCCTGTCCAGC
TTTAAGCACGTGATCAGAGCCCAGGTGTACGTGGAGGAGGTGCCTTGGAAGAG
GTTCGAGAAGAATGGCGTGAAGCACGTGCACGCCTTTATCTACACACCCACCG
GCACCCACTTCTGCGAGGTGGAGCAGATCAGAAATGGCCCTCCCGTGATCCAC
TCCGGCATCAAGGACCTGAAGGTGCTGAAGACCACCCAGAGCGGCTTCGAGG
GCTTCATCAAGGACCAGTTCACCACCCTGCCCGAGGTGAAGGATAGATGCTTC
GCCACACAGGTGTACTGTAAGTGGAGATACCACCAGGGCAGAGACGTGGACTT
CGAGGCCACATGGGACACAGTGAGAAGCATCGTGCTGCAGAAGTTTGCCGGC
CCTTACGACAAGGGCGAGTACAGCCCCTCTGTGCAGAAGACCCTCTATGATAT
CCAGGTGCTCTCCCTGAGCCGAGTTCCTGAGATAGAAGATATGGAAATCAGCC
TGCCAAACATTCACTACTTCAATATAGACATGTCCAAAATGGGTCTGATCAACAA
GGAAGAGGTCTTGCTGCCATTAGACAATCCATATGGAAAAATTACTGGTACAGT
CAAGAGGAAGTTGTCTTCAAGACTGTGACGGCTATTATGCGTTACCGGCGAGA
CGCTACGGACTT
SEQ ID NO. 51 IRES9-IL2-PBC
AAAATCCGTTGACCTTAAACGGTCGTGTGGGTTCAAGTCCCTCCACCCCCACG
CCGGAAACGCAATAGCCGACTGGAGTTCTGTGACAGGAGCCACCATGTACAGG
ATGCAACTCCTGTCTTGCATTGCACTAAGTCTTGCACTTGTCACGAATTCGGCC
CACTACAGGAATGATTACAAGAAGAACGATGAGGTGGAGTTCGTGAGAACCGG
CTACGGCAAGGACATGATCAAGGTGCTGCACATCCAGAGAGACGGCAAGTACC
ACAGCATCAAGGAGGTGGCCACCTCCGTGCAGCTGACACTGAGCTCCAAGAAG
GATTACCTGCACGGCGATAATAGCGATGTGATCCCCACAGACACCATCAAGAA
CACAGTGAACGTGCTGGCCAAGTTTAACGGCATCAAGTCCATCGAGACATTTGC
CGTGACCATCTGTGAGCACTTCCTGTCCAGCTTTAAGCACGTGATCAGAGCCCA
GGTGTACGTGGAGGAGGTGCCTTGGAAGAGGTTCGAGAAGAATGGCGTGAAG
CACGTGCACGCCTTTATCTACACACCCACCGGCACCCACTTCTGCGAGGTGGA
GCAGATCAGAAATGGCCCTCCCGTGATCCACTCCGGCATCAAGGACCTGAAGG
TGCTGAAGACCACCCAGAGCGGCTTCGAGGGCTTCATCAAGGACCAGTTCACC
ACCCTGCCCGAGGTGAAGGATAGATGCTTCGCCACACAGGTGTACTGTAAGTG
GAGATACCACCAGGGCAGAGACGTGGACTTCGAGGCCACATGGGACACAGTG
AGAAGCATCGTGCTGCAGAAGTTTGCCGGCCCTTACGACAAGGGCGAGTACAG
CCCCTCTGTGCAGAAGACCCTCTATGATATCCAGGTGCTCTCCCTGAGCCGAG
TTCCTGAGATAGAAGATATGGAAATCAGCCTGCCAAACATTCACTACTTCAATAT
AGACATGTCCAAAATGGGTCTGATCAACAAGGAAGAGGTCTTGCTGCCATTAGA
CAATCCATATGGAAAAATTACTGGTACAGTCAAGAGGAAGTTGTCTTCAAGACT
GTGACGGCTATTATGCGTTACCGGCGAGACGCTACGGACTT
SEQ ID NO. 52 forward primer
GGCCAGTGAATTGTAATACG
SEQ ID NO. 53 reverse primer
AACTCCGTAGCGTCTCGCCG
SEQ ID NO. 54 5′ homology arm
CGCCGGAAACGCAATAGCCG
SEQ ID NO. 55 3′ homology arm
CGGCTATTATGCGTTACCGGCG
SEQ ID NO. 56 IL2 signal peptide
ATGTACAGGATGCAACTCCTGTCTTGCATTGCACTAAGTCTTGCACTTGTCACG
AATTCG
SEQ ID NO. 57 PBC-F1
GCCACCATGTACAGGATGCA
SEQ ID NO. 58 PBC-R1
TCACAGTCTTGAAGACAACT
SEQ ID NO. 59 pCIRC-F2
AGTTGTCTTCAAGACTGTGACGGCTATTATGCGTTACCGG
SEQ ID NO. 60 pCIRC-R2
TGCATCCTGTACATGGTGGCTTTGCTGTATTCAACTTAAC
SEQ ID NO. 61 urate oxidase coding fragment PBC
ATGGCCCATTATAGAAACGATTACAAGAAGAATGATGAGGTGGAGTTCGTGAGG
ACCGGCTATGGCAAGGACATGATCAAGGTGCTGCACATTCAGCGGGACGGCAA
GTACCACTCCATCAAGGAGGTGGCTACTTCCGTCCAGCTGACCCTGAGCAGCA
AGAAGGACTATCTGCATGGGGATAACAGCGATGTTATCCCCACAGATACCATCA
AGAACACCGTGAATGTCCTTGCCAAGTTCAAGGGCATCAAGTCCATTGAGACTT
TTGCAGTGACGATCTGCGAGCACTTCCTGAGTAGCTTCAAGCACGTGATCCGC
GCCCAGGTGTATGTGGAGGAGGTGCCGTGGAAGCGGTTCGAGAAGAACGGCG
TGAAGCACGTGCACGCCTTCATATACACCCCCACCGGGACTCACTTCTGCGAG
GTGGAGCAGATCCGGAATGGACCCCCCGTGATCCACTCTGGGATCAAGGACCT
GAAGGTCCTGAAGACCACCCAGAGTGGATTCGAGGGGTTCATTAAGGATCAGT
TCACCACCTTGCCAGAGGTGAAGGACCGGTGCTTCGCGACCCAGGTGTACTGC
AAGTGGCGGTACCACCAGGGTCGCGACGTGGATTTTGAGGCTACTTGGGACAC
TGTTCGCAGCATCGTCCTGCAGAAGTTCGCCGGTCCTTACGACAAGGGGGAGT
ACTCCCCCTCCGTGCAGAAGACTCTGTACGACATCCAGGTCCTCACTCTCGGG
CAGGTGCCCGAGATTGAGGACATGGAGATCTCTCTGCCCAACATTCACTATCTG
AATATCGACATGTCTAAGATGGGCCTGATCAACAAGGAGGAGGTCCTGCTTCCT
CTTGACAATCCCTATGGGCGGATCACAGGGACTGTCAAGAGGAAGCTGACCTC
CCGCTTGTGA
SEQ ID NO. 62 urate oxidase coding fragment PBC
ATGGCCCACTATCGGAATGACTACAAGAAGAATGACGAGGTGGAGTTCGTGAGG
ACCGGCTATGGCAAGGACATGATCAAGGTGCTGCACATTCAGCGGGACGGCAA
GTACCACTCCATCAAGGAGGTGGCTACTTCCGTCCAGCTGACCCTGAGCAGCA
AGAAGGACTATCTGCATGGGGATAACAGCGATGTTATCCCCACAGATACCATCAA
GAACACCGTGAATGTCCTTGCCAAGTTCAAGGGCATCAAGTCCATTGAGACTTT
TGCAGTGACGATCTGCGAGCACTTCCTGAGTAGCTTCAAGCACGTGATCCGCG
CCCAGGTGTACGTGGAGGAGGTGCCATGGAAGCGGTTCGAGAAGAACGGCGT
GAAGCATGTGCACGCCTTCATCTACACCCCCACCGGGACTCACTTCTGCGAGG
TGGAGCAGATCCGGAATGGACCCCCCGTGATCCACTCTGGGATCAAGGACCTG
AAGGTCCTGAAGACCACCCAGAGTGGATTCGAGGGGTTCATTAAGGATCAGTTC
ACCACCTTGCCAGAGGTGAAGGACCGGTGCTTCGCGACCCAGGTGTACTGCAA
GTGGCGGTACCACCAGGGTCGCGACGTGGATTTTGAGGCTACTTGGGACACAG
TGCGCAGCATCGTCCTGCAGAAGTTCGCCGGTCCTTACGACAAGGGGGAGTAC
AGCCCCTCCGTGCAGAAGACCCTGTACGATATTCAGGTGCTGACCCTGGGGCA
GGTGCCTGAGATCGAGGACATGGAGATCAGCCTGCCCAACATTCACTACCTGAA
TATCGACATGAGCAAGATGGGTCTGATCAACAAGGAGGAGGTGCTGCTCCCCCT
TGACAACCCTTACGGCCGGATCACCGGCACCGTGAAGCGGAAGCTGACCAGCC
GGCTGTGA

What is claimed is:

1. A recombinant nucleic acid molecule for making a circular RNA, the circular RNA being capable of expressing a urate oxidase in cells, the recombinant nucleic acid molecule comprising elements operably linked to each other and arranged, in a 5′ to 3′ direction, in the following order:
(a) an intron fragment which includes a full-length intron;
(b) an E2 fragment which includes a downstream exon of the full-length intron;
(c) an internal ribosome entry site (IRES) fragment;
(d) a urate oxidase coding fragment; and
(e) an E1 fragment which includes an upstream exon of the full-length intron;
wherein the full-length intron, the downstream exon, and the upstream exon are from a same gene.

2. The recombinant nucleic acid molecule according to claim 1, wherein an amino acid sequence of the urate oxidase coding fragment has at least 95% similarity with any one of SEQ ID NOs. 1 and 3-8; or
a DNA sequence of the urate oxidase coding fragment has at least 95% similarity with SEQ ID NOs.9, 10, 61, or 62.

3. The recombinant nucleic acid molecule according to claim 1, further comprising a signal peptide element, which encodes a signal peptide that is configured to facilitate secreting the urate oxidase outside of the cells, wherein the signal peptide element is positioned between the IRES fragment and the urate oxidase coding fragment.

4. The recombinant nucleic acid molecule according to claim 3, wherein the signal peptide includes any one of: an interleukin-2 (IL-2) signal peptide, a human leukocyte antigen (HLA) signal peptide, a leucine-rich α-2 glycoprotein 1 (LRG1) signal peptide, a cholinergic receptor nicotinic alpha 1 subunit (CHRNA1) signal peptide, an apolipoprotein B (APOB) signal peptide, a cystatin D (CST5) signal peptide, a galactosylceramidase (GALC) signal peptide, a gelsolin (GSN) signal peptide, a glycoprotein Ib platelet subunit alpha (GP1BA) signal peptide, a granzyme B (GZMB) signal peptide, a SERPING1 signal peptide, an Interleukin-12 subunit alpha (IL-12A) signal peptide, an interleukin-2 (IL-10) signal peptide, an interleukin 1 receptor-like 1 (IL1RL1) signal peptide, an insulin receptor (INSR) signal peptide, a killer cell Immunoglobulin like receptor, two Ig domains and long cytoplasmic tail 1 (KIR2DL1) signal peptide, a kallikrein related peptidase 14 (KLK14) signal peptide, a lacritin (LACRT) signal peptide, and a lymphocyte activation gene-3 (LAG3) signal peptide.

5. The recombinant nucleic acid molecule according to claim 6, wherein an amino acid sequence of the signal peptide has at least 95% similarity with any one of SEQ ID NOs. 11-29.

6. The recombinant nucleic acid molecule according to claim 1, further comprising a signal positioning element, which encodes a signal positioning peptide that is configured for positioning the urate oxidase to a peroxisome, wherein the signal positioning element is positioned between the IRES fragment and the urate oxidase coding fragment.

7. The recombinant nucleic acid molecule according to claim 6, wherein an amino acid sequence of the signal positioning peptide has at least 97% similarity with SRL.

8. The recombinant nucleic acid molecule according to claim 1, wherein the IRES element is derived from a Taura syndrome virus, Triatoma virus, Theiler's murine encephalomyelitis virus, simian virus 40, Solenopsis invicta virus 1, Rhopalosiphum padi virus, reticuloendotheliosis virus, Poliovirus type 1, Plautia stali intestine virus, Kashmir bee virus, human rhinovirus 2, human immunodeficiency virus type 1, Homalodisca coagulata virus-1, Himetobi P virus, hepatitis C virus, hepatitis A virus, hepatitis B virus, foot-and-mouth disease virus, human enterovirus 71, equine rhinovirus, Ectropis obliqua picorna-like virus, Encephalomyocarditis virus (EMCV), Drosophila C virus, tobacco mosaic virus, cricket paralysis virus, bovine viral diarrhea virus 1, black queen cell virus, aphid lethal paralysis virus, avian encephalomyclitis virus, acute bee paralysis virus, Hibiscus Chlorotic Ringspot virus, hog cholera virus, salivary virus, Coxsackie virus, Parechovirus, simian picornavirus, turnip crinkle virus, Coxsackie virus B1 (CVB1), Coxsackie virus B2 (CVB2), or Coxsackie virus B3 (CVB3);
the IRES element is cloned from a gene coding a protein selected from a group consisting of: human FGF2, human SFTPA1, human AMLI/RUNXI, Drosophila antenna, human AQP4, human AT1R, human BAG-1, human BCL2, human BiP, human cIAP-1, human c-myc, human eIF4G, mouse NDST4L, human LEF1, mouse HIF1α, human n-myc, mouse Gtx, human p27KipI, human PDGF2/c-sis, human p53, human Pim-1, mouse Rbm3, Drosophila reaper, dog Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, and an aptamer of eIF4G; or
the IRES element includes ribosome recognition sequences pIRES1-pIRES10.

9. The recombinant nucleic acid molecule according to claim 1, wherein a DNA sequence of the IRES element has at least 95% similarity with any one of SEQ ID NOs. 30-40.

10. The recombinant nucleic acid molecule according to claim 1, wherein a DNA sequence of the IRES element has at least 95% similarity with SEQ ID NO. 38 or SEQ ID NO. 40.

11. The recombinant nucleic acid molecule according to claim 1, wherein the intron fragment includes an intron of the pre-tRNA_{Leu} gene of genus Anabaena;
the E2 fragment includes a downstream exon of the intron of the pre-tRNA_{Leu} gene of genus Anabaena; and
the E1 fragment includes an upstream exon of the intron of the pre-tRNA_{Leu} gene of genus Anabaena.

12. The recombinant nucleic acid molecule according to claim 11, wherein a nucleotide sequence of the intron fragment has at least 95% similarity with SEQ ID NO. 41, a nucleotide sequence of the E2 fragment has at least 95% similarity with any one of SEQ ID NO. 42 to SEQ ID NO. 45, AAAATCCG, AAAATC, AAAA, and AA, a nucleotide sequence of the E1 fragment has at least 95% similarity with any one sequence of SEQ ID NO. 46 to SEQ ID NO. 49, GGACTT, ACTT, TT, and CTT.

13. The recombinant nucleic acid molecule according to claim 1, further comprising a 5′ homology arm sequence and a 3′ homology arm sequence positioned between the E2 fragment and the E1 fragment.

14. The recombinant nucleic acid molecule according to claim 1, wherein the intron fragment is further preceded by a promoter which initiates in vitro transcription of the recombinant nucleic acid molecule.

15. A linear RNA which is produced based on the recombinant nucleic acid molecule according to claim 1.

16. A circular RNA which is produced based on the recombinant nucleic acid molecule according to claim 1.

17. A method for preparing a circular RNA based on the recombinant nucleic acid molecule according to claim 1, comprising:
obtaining a linear RNA by performing an in vitro transcription reaction on the recombinant nucleic acid molecule; and
allowing the linear RNA to self-circularize to produce the circular RNA.

18. The method according to claim 17, further comprising encapsulating the circular RNA by lipid nanoparticles (LNP).

19. The method according to claim 18, wherein the encapsulating the circular RNA by LNP further includes:
dissolving the LNP into ethyl alcohol to obtain a LNP solution;
dissolving the circular RNA into a sodium acetate solution to obtain a circular RNA solution; and
obtaining the LNP-encapsulated circular RNA by making the LNP solution and the circular RNA solution pass through a microfluidic device.

20. A method for reducing a uric acid in a subject or treating a disease with a high uric acid level in a subject, comprising:
administering the circular RNA according to claim 16, in a pharmaceutically acceptable amount, to the subject.
